(19) United States
(12) Patent Application Publication
(10) Pub. No.: US 2026/0193345 A1
(43) Pub. Date: Jul. 9, 2026

(54) METHODS OF TREATING OBESITY

(71) Applicant: THE UNIVERSITY OF FLORIDA RESEARCH FOUNDATION, INCORPORATED, Gainesville, FL (US)

(72) Inventors: Zachary Krumm, Gainesville, FL (US); Edgardo Rodriguez, Gainesville, FL (US); Hunter S. Futch, Gainesville, FL (US); Todd Eliot Golde, Decatur, GA (US)

(21) Appl. No.: 19/420,925

(22) Filed: Dec. 16, 2025

Related U.S. Application Data
(60) Provisional application No. 63/736,072, filed on Dec. 19, 2024.

(51) Int. Cl. C07K 16/26; A61P 3/04

(52) U.S. Cl. CPC C07K 16/26; A61P 3/04; C07K 2317/24; C07K 2317/622

(57) ABSTRACT

Disclosed herein are materials and methods for the treatment of obesity.

### CROSS REFERENCE TO RELATED APPLICATIONS

This application claims the benefit of priority to U.S. Provisional Application Ser. No. 63/736,072, entitled “METHODS OF TREATING OBESITY,” filed Dec. 19, 2024, the contents which is hereby incorporated by reference.

### STATEMENT OF GOVERNMENT SUPPORT

This invention was made with government support under Grant No. AG064942, awarded by the National Institutes of Health. The government has certain rights in the invention.

### FIELD OF THE INVENTION

The present disclosure is directed to anti-corticotropin-releasing factor (CRF) antibodies and the use of such antibodies for the treatment of disorders associated with hypothalamic-pituitary adrenal (HPA) axis activation (e.g. obesity).

### INCORPORATION BY REFERENCE OF MATERIALS SUBMITTED ELECTRONICALLY

This application contains, as a separate part of the disclosure, a Sequence Listing in computer readable form (Filename: 70820P_Seqlisting.txt; Size: 21,672 bytes; Created: Dec. 18, 2024), which is incorporated by reference in its entirety.

### BACKGROUND

Corticotropin-releasing factor (CRF), also known as corticotropin-releasing hormone, is a central coordinator of the neuroendocrine and behavioral response to stressful stimuli. CRF has been evolutionarily conserved in the vertebrate lineage from a time preceding the teleosts and tetrapods, over 550 million years. This conservation across species highlights the importance of CRF in the vertebrate response to stressful stimuli.

CRF signals through its receptor the CRHR1 to control hypothalamic-pituitary-adrenal (HPA) axis activation; CRF release from the paraventricular nucleus (PVN) of the hypothalamus acts on CRHR1 receptors in the anterior pituitary, causing Adrenocorticotropic Hormone (ACTH) release that stimulates the adrenal glands to produce and release glucocorticoids (GCs), namely cortisol in humans and corticosterone in rodents. Glucocorticoids freely diffuse throughout the body and act on high affinity mineralocorticoid (MR) and low affinity glucocorticoid (GR) receptors that are expressed in almost every vertebrate cell type. MR and GR signaling cause both rapid functional responses in many cell types in addition to long-standing effects through regulation of transcription via glucocorticoid-response elements (GREs). The response to GR and MR receptor activation leads to mobilization of resources and cellular adaptations that prepare the body to overcome a stressor.

In the absence of a stressful stimulus, the PVN receives input from the suprachiasmatic nucleus (SCN) of the hypothalamus and responds to melatonin levels, altering CRF release throughout the day based on these inputs. These daily fluctuations in CRF release in turn produce the circadian rhythm of GC release, with peak levels of GCs being present at awakening and trough levels before returning to sleep.

In addition to its effects on the HPA Axis, CRF and the CRHR1 are widely expressed in many regions of the brain implicated in cognition and anxiety, including the neocortex, hippocampus, amygdala, and locus coeruleus. CRF has been shown to have region specific effects on anxiety and is best described as neuroregulatory: augmenting the activity of CRHR1 expressing neurons but not serving as a classical neurotransmitter.

### SUMMARY

Methods of treating a disorder associated with HPA axis activation (e.g., obesity) are contemplated. In some embodiments, the antibody comprises administering an anti-corticotropin-releasing factor (CRF) antibody (or antigen-binding fragment thereof) to a subject in need thereof. In some embodiments, the antibody or antigen binding fragment thereof comprises a set of six CDRs set forth in SEQ ID NOs: 12-17 or SEQ ID NOs: 4-9. In some embodiments, the antibody or antigen binding fragment thereof comprises a heavy chain variable region comprising an amino acid sequence set forth in SEQ ID NO: 19 or SEQ ID NO: 11. In some embodiments, the antibody or antigen binding fragment thereof comprises a light chain variable region comprising an amino acid sequence set forth in SEQ ID NO: 18 or 10.

In some embodiments, the antibody or antigen binding fragment thereof is a monoclonal antibody. In some embodiments, the antibody or antigen binding fragment thereof is a humanized antibody. In some embodiments, the antibody or antigen binding fragment thereof comprises two heavy chains and two light chains. In some embodiments, the antibody is an IgG. In some embodiments, the antigen binding fragment is a Fab fragment or an scFv. In some embodiments, the scFv comprises an amino acid sequence set forth in SEQ ID NO: 22 or SEQ ID NO: 23.

### BRIEF DESCRIPTION OF THE FIGURES

FIGS. 1A-1B demonstrate that Antibody B suppressed basal and stress-induced HPA-Axis output from batch-to-batch. C57BL6/J×C3H/HeJ male mice were single housed for 1 week prior to injection with 20 mg/kg several batches of anti-CRF antibody (Antibody B), and IgG1 isotype-matched control, or no injection (n=3-4 animals/group). 48 hours after injection, animals were subjected to 30 minutes of restraint stress, where blood was collected for corticosterone (CORT) measurements at 0, 30, 60 and 90 minutes after stress initiation (FIG. 1A). Basal CORT levels were measured immediately upon the animals being placed within the restrainer, within 60 seconds of being removed from the home cage (FIG. 1B). Values are mean+/−SEM. (*, P<0.05; **, P<0.01).

FIGS. 2A-2F show that Antibody B treatment lowered plasma CORT levels and improves weight and body composition in C57BL6/J male mice. 2-month-old C57BL6/J male mice were placed on high-fat diet for 4 weeks prior to receiving 1 of 4 treatments: no injection/handling control (circle), weekly 12.5 mg/kg IgG1 Control injection (diamond), a single 25 mg/kg dose on week 1 followed 12.5 mg/kg IgG1 control injections weekly (square), and a 25 mg/kg loading dose followed by weekly 12.5 mg/kg weekly injections of Antibody B (triangle (n=9-10 animals per cohort). Animals were then maintained on high-fat diet for 4 months. (FIG. 2A) Weight gain trajectories of each cohort. (FIG. 2B) Weekly food consumption per animal. (FIG. 2C) Change in percent (%) of body mass that is lean mass relative to point of randomization. (FIG. 2D) Change in percent (%) of body mass that is fat mass relative to point of randomization. (FIG. 2E) Area under the curve (AUC) of post-intervention IPGTT minus pre-intervention of IPGTT. (FIG. 2F) CORT levels immediately prior to first injection, at experimental midpoint, and at experimental endpoint. Values are mean+/−SEM. (FIGS. 2A-2D, Repeat measures ANOVA with Dunnett correction for multiple comparisons; FIGS. 2E-2F, One-way ANOVA with Welch correction and Brown-Forsythe correction for multiple comparisons; *, P<0.05; **, P<0.01; ***, P<0.001; ****, P<0.0001). Summary of statistical tests can be viewed in Table 1.

FIGS. 3A-3D demonstrate that Antibody B improved weight trajectories and body composition in high-fat diet C57BL/J×C3H/H3J male and female mice. 7-month-old C57BL6/J×C3H/HeJ hybrid strain (B6C3) male and female mice were maintained on high-fat diet for 4 months. Animals were then randomized to receive either 12.5 mg/kg weekly Antibody B injections (circle), a single 25 mg/kg Antibody B injection followed by 12.5 mg/kg weekly IgG1 control injections (square), or 12.5 mg/kg (triangle). Animals were maintained on these treatments and continued to receive high-fat diet for 4 months. (FIGS. 3A-3B) Gross weights throughout this study in males (FIG. 3A) and females (FIG. 3B). (FIGS. 3C-3D) Relative change from point of randomization in males (FIG. 3C) and females (FIG. 3D). Values are mean+/−SEM. (Repeat measures ANOVA with Dunnett correction for multiple comparisons; *, P<0.05; **, P<0.01; ***, P<0.001; ****, P<0.0001). Summary of statistical tests can be viewed in Table 2 and Table 3 (below).

FIGS. 4A-4F demonstrate that Antibody B treatment shifts the distribution of weight changes from point of randomization to endpoint. Percent (%) weight change relative to point of randomization of cohorts are plotted in ascending order (Waterfall Plots). (FIGS. 4A-4C) Male weekly Antibody B (FIG. 4A) (n=19 mice), Single dose Antibody B (FIG. 4B) (n=12 mice), and IgG1 control (FIG. 4C) (n=17 mice). (FIGS. 4D-4F) Female weekly Antibody B (FIG. 4D) (n=21 mice), Single dose Antibody B (FIG. 4E) (n=14 mice), and IgG1 control (FIG. 4F) (n=19 mice).

FIGS. 5A-5H show that Antibody B treatment improves body composition on an absolute and percentage-wise basis in both male and female B6C3 mice. Body composition was assessed by EchoMRI the week prior to randomization and then every 4 weeks afterwards. (FIG. 5A) Absolute total fat mass in male cohorts at each timepoint. (FIG. 5B) Percentage of total body weight that is fat mass in male cohorts at each timepoint. (FIG. 5C) Absolute total lean mass in male cohorts at each timepoint. (FIG. 5D) Percentage of total body weight that is lean mass in male cohorts at each timepoint. (FIG. 5E) Absolute total fat mass in female cohorts at each timepoint. (FIG. 5F) Percentage of total body weight that is fat mass in female cohorts at each timepoint. (FIG. 5G) Absolute total lean mass in female cohorts at each timepoint. (FIG. 5H) Percentage of total body weight that is lean mass in female cohorts at each timepoint. Values are mean+/−SEM. (Repeat measures ANOVA with Dunnett correction for multiple comparisons; *, P<0.05; **, P<0.01; ***, P<0.001; **** P<0.0001). Summary of statistical tests can be viewed in Table 2 and Table 3.

FIGS. 6A-6L demonstrate that Antibody B treatment impacted the distribution of fat storage and decreases gross liver mass in B6C3 male and female mice. Post-mortem mesenteric fat, subcutaneous/inguinal fat, and liver masses were assessed. Fasting plasma glucose was assessed at experimental endpoint, while plasma CORT levels were taken at the time of randomization, as the midpoint of the experimental window, and at endpoint. (FIGS. 6A-6B) Ratio of mass of the subcutaneous adipose fat pad versus the mesenteric fat pad in males (FIG. 6A) and females (FIG. 6B). (FIGS. 6C-6D) Endpoint gross liver weights in males (FIG. 6C) and females (FIG. 6D). (FIGS. 6E-6F) Association between bodyweight and liver mass in males (FIG. 6E) and females (FIG. 6F). (FIGS. 6G-6H) Association between mesenteric fat pad mass and liver mass in males (FIG. 6G) and females (FIG. 6H). (FIG. 6I) Fasting plasma glucose at endpoint in male and female cohorts. (FIG. 6J) Schematic of relative glucocorticoid receptor expression in visceral (mesenteric) fat pads versus subcutaneous fat pads. (FIG. 6K) Representative images of trichrome staining in weekly dose versus IgG1 control cohorts (note—the results are preliminary and should not be considered indicative at this time). (FIG. 6L) CORT levels immediately prior to randomization, at the midpoint, and at the endpoint of the 4-month experimental window. Values are mean+/−SEM. (FIGS. 6A-6D, FIG. 6I, FIG. 6L, One-way ANOVA with Welch correction and Brown-Forsythe correction for multiple comparisons; FIGS. 6E-6H, simple linear regression with 95% confidence intervals; *, P<0.05; **, P<0.01; ***, P<0.001; ****, P<0.0001). Summary of statistical tests can be viewed in Table 2 and Table 3.

FIGS. 7A-7D show that weekly Antibody B injections correlated with increases in gross quadriceps and gastrocnemius weights relative to bodyweight at experimental endpoint. Gastrocnemius and quadriceps muscle were dissected and weighed at experimental endpoint. (FIG. 7A) Gross mass of quadriceps muscle in male cohorts, relative to bodyweight. (FIG. 7B) Gross mass of gastrocnemius muscle in male cohorts, relative to bodyweight. (FIG. 7C) Gross mass of quadriceps muscle in female cohorts, relative to bodyweight. (FIG. 7D) Gross mass of gastrocnemius muscle in female cohorts, relative to bodyweight. Values are mean+/−SEM. (One-way ANOVA with Welch correction and Brown-Forsythe correction for multiple comparisons; *, P<0.05; **, P<0.01; ***, P<0.001; ****, P<0.0001, non-significant data not shown).

FIGS. 8A-8D reveal that HPA-Axis suppression improved glucose handling assessed by oral glucose tolerance test. At the experimental endpoint, both male and female cohorts were fasted for 5 hours prior to receiving an oral gavage of 2 g/kg dextrose. Plasma glucose levels were measured immediately prior to gavage and 15, 30, 60, and 120 minutes after gavage. (FIG. 8A) Plasma glucose at individual timepoints in male cohorts (weekly dose, n=16 mice; Single Dose, n=9 mice; IgG1 Control, n=15 mice; Stressed weekly dose, n=9 mice; stress IgG1 control, n=8 mice). (FIG. 8B) Area under the curve for cohorts in (FIG. 8A). (FIG. 8C) Plasma glucose at individual timepoints in female cohorts (weekly dose, n=16 mice; Single dose, n=9 mice; IgG1 Control, n=16 mice). (FIG. 8D) Area under the curve for cohorts in (FIG. 8C). Values are mean+/−SEM. (One-way ANOVA with Welch correction and Brown-Forsythe correction for multiple comparisons; *, P<0.05; **, P<0.01; ***, P<0.001; ****, P<0.0001, non-significant data not shown).

FIGS. 9A-9D show that HPA-Axis suppression improved insulin sensitivity as assessed by insulin tolerance test in B6C3 mice. At the experimental endpoint, both male and female cohorts were fasted for 5 hours prior to receiving an IP injection of 1 U/kg of Humalin-R insulin. Plasma glucose levels were measured immediately prior to gavage and 15, 30, 60, and 90 minutes after gavage. (FIG. 9A) Plasma glucose at individual timepoints in male cohorts (weekly dose, n=16 mice; Single Dose, n=9 mice; IgG1 Control, n=15 mice; Stressed weekly dose, n=9 mice; stress IgG1 control, n=8 mice). (FIG. 9B) Area under the curve for cohorts in (FIG. 9A). (FIG. 9C) Plasma glucose at individual timepoints in female cohorts (weekly dose, n=16 mice; Single dose, n=9 mice; IgG1 Control, n=16 mice). (FIG. 9D) Area under the curve for cohorts in (FIG. 9C). Values are mean+/−SEM. (One-way ANOVA with Welch correction and Brown-Forsythe correction for multiple comparisons; *, P<0.05; **, P<0.01; ***, P<0.001; ****, P<0.0001, non-significant data not shown).

FIGS. 10A-10D show that HPA-Axis suppression blunts weight gain trajectory in MC4r and leptin knockout models of obesity. Leptin and MC4r wild-type, heterozygous, and knockout mice were maintained on a normal chow diet and injected with either 12.5 mg/kg Antibody B or an IgG1 control antibody from 6-16 weeks of age. (FIG. 10A) Weight trajectories of male MC4r knockout (Antibody B cohort, n=6; IgG1 control cohort, n=6), heterozygotes (Antibody B, n=8; IgG1 control, n=6), and wild type (Antibody B, n=5, IgG1 control, n=6) mice. (FIG. 10B) Weight trajectories of female MC4r knockout (Antibody B cohort, n=6; IgG1 control cohort, n=5), heterozygotes (Antibody B, n=5; IgG1 control, n=5), and wild type (Antibody B, n=5, IgG1 control, n=7) mice. (FIG. 10C) Weight trajectories of male leptin knockout (Antibody B cohort, n=7; IgG1 control cohort, n=6), heterozygotes (Antibody B, n=5; IgG1 control, n=4), and wild type (Antibody B, n=5, IgG1 control, n=4) mice. (FIG. 10D) Weight trajectories of female leptin knockout (Antibody B cohort, n=7; IgG1 control cohort, n=6), heterozygotes (Antibody B, n=8; IgG1 control, n=12), and wild type (Antibody B, n=3, IgG1 control, n=4) mice. Values are mean+/−SEM. (Repeated measures ANOVA with Dunnett correction for multiple comparisons; *, P<0.05; **, P<0.01; ***, P<0.001; ****, P<0.0001, non-significant data not shown).

FIGS. 11A-11B show that HPA-Axis suppression decreased gross liver mass and increases muscle mass in MC4r and leptin knockout mice. (FIG. 11A) Post-mortem gross liver weight in male and female MC4r and leptin knockout mice. (FIG. 11B) Post-mortem gross weights of quadriceps and gastrocnemius muscle in male and female MC4r and leptin knockout mice. (Multiple student T-Tests with Welch correction and Bonferroni correction for multiple comparisons; *, P<0.05; **, P<0.01; ***, P<0.001; ****, P<0.0001; ns, P>0.05).

FIGS. 12A-12J demonstrates that HPA-Axis suppression induced significant weight reduction and improvements in body composition in both male and female mice within 6 weeks of treatment. 21-22-month-old male and female mice were injected with 12.5 mg/kg of Antibody B or IgG1 control weekly for 6 weeks. (FIGS. 12A-12B) Weight trajectories for male (Antibody B, n=10; control, n=8) (FIG. 12A) and female (Antibody B, n=15; control, n=13) (FIG. 12B) mice. (FIGS. 12C-12F) Total and percent fat mass at endpoint in male (FIGS. 12C-12D) and female (FIGS. 12E-12F) mice. (FIGS. 12G-12J) Total and percent lean mass at endpoint in male (FIGS. 12G-12H) and female (FIGS. 121-12J) mice. (Student T-Tests with Welch correction; *, P<0.05; **, P<0.01; ***, P<0.001; ****, P<0.0001; ns, P>0.05).

FIGS. 13A-13B show that AAV-CRF injection modulated body weight and glucose metabolism in PS19 P301S tau-transgenic mice with variable impacts on fasting plasma glucose. PS19 P301S tau-transgenic mice and non-transgenic littermates were injected with AAV-CRF or an AAV-GFP control. (FIG. 13A) Endpoint weights of male (Transgenic CRF, n=5; Transgenic GFP, n=5; non-Transgenic CRF, n=7; non-Transgenic GFP, n=6) and females (Transgenic CRF, n=6; Transgenic GFP, n=6; non-Transgenic CRF, n=4; non-Transgenic GFP, n=6). (FIG. 13B) Animals were fasted for 5 hours prior to assessing fasting glucose near the experimental endpoint in the cohorts described in (FIG. 13A). (One-way ANOVA with Welch correction and Brown-Forsythe correction for multiple comparisons; *, P<0.05; **, P<0.01; ***, P<0.001; ns, P>0.05).

FIGS. 14A-14B demonstrate that AAV-CRF injections significantly increased plasma CORT levels in PS19 tau mice, while Antibody B injection significantly suppress plasma CORT levels in JNPL3 tau mouse models. To confirm that findings in PS19 tau-transgenic animals injected with AAV-CRF were consistent with elevated levels of glucocorticoids, plasma CORT was measured near the experimental endpoint. In a separate experiment (because JNPL3 animals were to be used for subsequent experiments), animals were injected with Antibody B, an IgG1 control, or no injection to determine if HPA-Axis suppression could be achieved in these animals. (FIG. 14A) Plasma CORT levels in male and female PS19 tau-transgenic mice injected with either AAV-GFP (left) or AAV-CRF (right) at day P0. (FIG. 14B) Plasma CORT levels in female (n=5) and male (n=5) 4-month-old homozygous JNPL3 P301L tau-transgenic mice injected with a 20 mg/kg dose of Antibody B, female (n=4) and male (n=4) animals injected with 20 mg/kg of an IgG1 control, or a male (n=6) that received no injection. (One-way ANOVA with Welch correction and Brown-Forsythe correction for multiple comparisons; *, P<0.05; **, P<0.01; ***, P<0.001; **** P<0.0001; ns, P>0.05).

FIGS. 15A-15B show that Antibody B treatment slowed muscle and adipose tissue atrophy in chow-diet JNPL3 tau model. JNPL3 transgenic animals were treated with 12.5 mg/kg weekly of Antibody B or an IgG1 control antibody beginning at 7 months (females) or 9 months (males). Animals were treated for 3 months. The female experiment also included a cohort that received Antibody B for the first 1.5 months of treatment and then IgG1 control antibody for the second 1.5 months. (FIG. 15A) Total gross fat mass (left) and lean mass (right) at the experimental endpoint of male JNPL3 animals treated with Antibody B (black shading, n=22 animals) or IgG1 control (gray shading, n=20 animals). (FIG. 15B) Total gross fat mass (left) and lean mass at the experimental endpoint of JNPL3 animals treated with Antibody B for all 3 months (black shading, n=21 animals), 1.5 months of Antibody B (light gray shading, n=17 animals), or an IgG1 control throughout (dark gray shading, n=20 animals). (Males, Student T-Tests with Welch correction; Females, One-way ANOVA with Welch correction and Brown-Forsythe correction for multiple comparisons; *, P<0.05; **, P<0.01; ***, P<0.001; ****, P<0.0001; ns, P>0.05).

### DETAILED DESCRIPTION

The present disclosure is based in part on the discovery that in vivo administration of an anti-corticotropin releasing factor (CRF) antibody inhibits a cortisol response to acute stress.

Corticotropin-releasing factor (CRF) is a 41-amino acid peptide (SEEPPISLDLTFHLLREVLEMARAEQLAQQAHSNRKLMEII (SEQ ID NO: 1)) derived from a 196-amino acid preprohormone (Genbank Accession No. EAW86897.1) encoded by the gene CRF. CRF is secreted by the paraventricular nucleus (PVN) of the hypothalamus in response to stress. Increased CRF production has been observed to be associated with Alzheimer's disease and major depression (Raadsheer et al., Am J Psychiatry. 152 (9): 1372-6, 1995), and autosomal recessive hypothalamic corticotropin deficiency has multiple (and potentially fatal) metabolic consequences, including hypoglycemia.

In some or any embodiments, the antibody (or antigen binding fragment) binds to CRF of SEQ ID NO: 1, or a naturally occurring variant thereof, with an affinity (Kd) of less than or equal to 1×10⁻⁷ M, less than or equal to 1×10⁻⁸ M, less than or equal to 1×10⁻⁹ M, less than or equal to 1×10⁻¹⁰ M, less than or equal to 1×10⁻¹¹ M, or less than or equal to 1×10⁻¹² M, or ranging from 1×10⁻⁹ to 1×10⁻¹⁰, or ranging from 1×10⁻¹² to about 1×10⁻¹³. Affinity is determined using a variety of techniques, examples of which include an affinity ELISA assay and a surface plasmon resonance (BIAcore) assay.

In some or any embodiments, the antibody (or antigen binding fragment thereof) binds to a CRF peptide comprising amino acids 1-21 of SEQ ID NO: 1, with any of the affinities described above. Alternatively, or in addition, the antibody (or antigen binding fragment thereof) binds to a CRF peptide comprising amino acids 1-15 of SEQ ID NO: 1, with any of the affinities described above.

The term “antibody” refers to an intact immunoglobulin molecule (including polyclonal, monoclonal, chimeric, humanized, and/or human versions having full length heavy and/or light chains). The antibody may be any type of antibody, i.e., immunoglobulin, known in the art. In exemplary embodiments, the antibody is an antibody of class or isotype IgA, IgD, IgE, IgG, or IgM. In exemplary embodiments, the antibody described herein comprises one or more alpha, delta, epsilon, gamma, and/or mu heavy chains. In exemplary embodiments, the antibody described herein comprises one or more kappa or light chains. In exemplary aspects, the antibody is an IgG antibody and optionally is one of the four human subclasses: IgG1, IgG2, IgG3 and IgG4. Also, the antibody in some embodiments is a monoclonal antibody. In other embodiments, the antibody is a polyclonal antibody. In some aspects, the antibody is a chimeric or a humanized antibody. The term “humanized” when used in relation to antibodies refers to antibodies having at least CDR regions from a non-human source and which are engineered to have a structure and immunological function more similar to true human antibodies than the original source antibodies. For example, humanizing can involve grafting CDRs from a non-human antibody, such as a mouse antibody, into a human antibody framework. Humanizing also can involve select amino acid substitutions to make a non-human sequence look more like a human sequence.

In some aspects, the antibody is a Humaneered™ antibody. Humaneering technology converts non-human antibodies into engineered human antibodies. Humaneered™ antibodies have high affinity and are highly similar to human germline antibody sequences. See, e.g., Tomasevic et al., Growth Factors 32:223-235 (2014).

“Specifically binds” as used herein means that the antibody (or antigen binding fragment) preferentially binds an antigen (CRF peptide) over other proteins. In some embodiments, “specifically binds” means the antibody has a higher affinity for the antigen than for other proteins. Antibodies that specifically bind an antigen may have a binding affinity for the antigen of less than or equal to 1×10⁻⁷ M, less than or equal to 2×10⁻⁷ M, less than or equal to 3×10⁻⁷ M, less than or equal to 4×10⁻⁷ M, less than or equal to 5×10⁻⁷ M, less than or equal to 6×10⁻⁷ M, less than or equal to 7×10⁻⁷ M, less than or equal to 8×10⁻⁷ M, less than or equal to 9×10⁻⁷ M, less than or equal to 1×10⁻⁸ M, less than or equal to 2×10⁻⁸ M, less than or equal to 3×10⁻⁸ M, less than or equal to 4×10⁻⁸ M, less than or equal to 5×10⁻⁸ M, less than or equal to 6×10⁻⁸ M, less than or equal to 7×10⁻⁸ M, less than or equal to 8×10⁻⁸ M, less than or equal to 9×10⁻⁸ M, less than or equal to 1×10⁻⁹ M, less than or equal to 2×10⁻⁹ M, less than or equal to 3×10⁻⁹ M, less than or equal to 4×10⁻⁹ M, less than or equal to 5×10⁻⁹ M, less than or equal to 6×10⁻⁹ M, less than or equal to 7×10⁻⁹ M, less than or equal to 8×10⁻⁹ M, less than or equal to 9×10⁻⁹ M, less than or equal to 1×10⁻¹⁰ M, less than or equal to 2×10⁻¹⁰ M, less than or equal to 3×10⁻¹⁰ M, less than or equal to 4×10⁻¹⁰ M, less than or equal to 5×10⁻¹⁰ M, less than or equal to 6×10⁻¹⁰ M, less than or equal to 7×10⁻¹⁰ M, less than or equal to 8×10⁻¹⁰ M, less than or equal to 9×10⁻¹⁰ M, less than or equal to 1×10⁻¹¹ M, less than or equal to 2×10⁻¹¹ M, less than or equal to 3×10⁻¹¹ M, less than or equal to 4×10⁻¹¹ M, less than or equal to 5×10⁻¹¹ M, less than or equal to 6×10⁻¹¹ M, less than or equal to 7×10⁻¹¹ M, less than or equal to 8×10⁻¹¹ M, less than or equal to 9×10⁻¹¹ M, less than or equal to 1×10⁻¹² M, less than or equal to 2×10⁻¹² M, less than or equal to 3×10⁻¹² M, less than or equal to 4×10⁻¹² M, less than or equal to 5×10⁻¹² M, less than or equal to 6×10⁻¹² M, less than or equal to 7×10⁻¹² M, less than or equal to 8×10⁻¹² M, or less than or equal to 9×10⁻¹² M. It will be appreciated that ranges having the values above as end points is contemplated in the context of the disclosure. For example, the antibody or antigen binding fragment thereof may bind CRF of SEQ ID NO: 1 with an affinity of about 1×10⁻⁷ M to about 9×10⁻¹² M or an affinity of 1×10⁻⁹ to about 9×10⁻¹².

“CDR” refers to the complementarity determining region within antibody variable sequences. There are three CDRs in each of the variable regions of the heavy chain and the light chain, which are designated CDR1, CDR2 and CDR3, for each of the variable regions. The term “set of six CDRs” as used herein refers to a group of three CDRs that occur in the light chain variable region and heavy chain variable region, which are capable of binding the antigen. The exact boundaries of CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262 (5): 73245 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

CDRs are obtained by, e.g., constructing polynucleotides that encode the CDR of interest and expression in a suitable host cell. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)).

In various aspects, the antibody (or antigen binding fragment thereof) comprises at least one CDR sequence having at least 75% identity (e.g., at least 75%, 80%, 85%, 90%, 95% or 100% identity) to a CDR selected from CDR-H1, CDR-H2, CDR-H3, CDR-L1, CDR-L2, and CDR-L3 wherein CDR-H1 has the sequence given in SEQ ID NO: 4, CDR-H2 has the sequence given in SEQ ID NO: 5, CDR-H3 has the sequence given in SEQ ID NO: 6, CDR-L1 has the sequence given in SEQ ID NO: 7, CDR-L2 has the sequence given in SEQ ID NO: 8 and CDR-L3 has the sequence given in SEQ ID NO: 9. The anti-CRF antibody, in various aspects, comprises two of the CDRs, three of the CDRs, four of the CDRs, five of the CDRs or all six of the CDRs. In a preferred embodiment, the anti-CRF antibody comprises a set of six CDRs as follows: CDR-H1 of SEQ ID NO: 4, CDR-H2 of SEQ ID NO: 5, CDR-H3 of SEQ ID NO: 6, CDR-L1 of SEQ ID NO: 7, CDR-L2 of SEQ ID NO: 8 and CDR-L3 of SEQ ID NO: 9.

In various aspects, the antibody (or antigen binding fragment thereof) comprises at least one CDR sequence having at least 75% identity (e.g., at least 75%, 80%, 85%, 90%, 95% or 100% identity) to a CDR selected from CDR-H1, CDR-H2, CDR-H3, CDR-L1, CDR-L2, and CDR-L3 wherein CDR-H1 has the sequence given in SEQ ID NO: 12, CDR-H2 has the sequence given in SEQ ID NO: 13, CDR-H3 has the sequence given in SEQ ID NO: 14, CDR-L1 has the sequence given in SEQ ID NO: 15, CDR-L2 has the sequence given in SEQ ID NO: 16 (KVS) and CDR-L3 has the sequence given in SEQ ID NO: 17. The anti-CRF antibody, in various aspects, comprises two of the CDRs, three of the CDRs, four of the CDRs, five of the CDRs or all six of the CDRs. In a preferred embodiment, the anti-CRF antibody comprise a set of six CDRs as follows: CDR-H1 of SEQ ID NO: 12, CDR-H2 of SEQ ID NO: 13, CDR-H3 of SEQ ID NO: 14, CDR-L1 of SEQ ID NO: 15, CDR-L2 of SEQ ID NO: 16 (KVS) and CDR-L3 of SEQ ID NO: 17.

In some or any embodiments, the antibody comprises a light chain variable region comprising an amino acid sequence having at least 75% identity (e.g., at least 75%, 80%, 85%, 90%, 95% or 100% identity) to the amino acid sequence set forth in SEQ ID NO: 10 and/or a heavy chain variable region comprising an amino acid sequence having at least 75% identity (e.g., at least 75%, 80%, 85%, 90%, 95% or 100% identity) to the amino acid sequence set forth in SEQ ID NO: 11. In various aspects, the difference in the sequence compared to SEQ ID NO: 10 or 11 lies outside the CDR region in the corresponding sequences. In some or any embodiments, the antibody (or antigen binding fragment) comprises a light chain variable region comprising an amino acid sequence set forth in SEQ ID NO: 10 and a heavy chain variable region comprising an amino acid sequence set forth in SEQ ID NO: 11. In some or any embodiments, the antibody (or antigen binding fragment) comprises a light chain variable region encoded by the nucleotide sequence set forth in SEQ ID NO: 22. In some or any embodiments, the antibody (or antigen binding fragment) comprises a heavy chain variable region encoded by the nucleotide sequence set forth in SEQ ID NO: 23.

In some or any embodiments, the antibody comprises a light chain variable region comprising an amino acid sequence having at least 75% identity (e.g., at least 75%, 80%, 85%, 90%, 95% or 100% identity) to the amino acid sequence set forth in SEQ ID NO: 18 and/or a heavy chain variable region comprising an amino acid sequence having at least 75% identity (e.g., at least 75%, 80%, 85%, 90%, 95% or 100% identity) to the amino acid sequence set forth in SEQ ID NO: 19. In various aspects, the difference in the sequence compared to SEQ ID NO: 18 or 19 lies outside the CDR region in the corresponding sequences. In some or any embodiments, the antibody (or antigen binding fragment thereof) comprises a light chain variable region comprising an amino acid sequence set forth in SEQ ID NO: 18 and a heavy chain variable region comprising an amino acid sequence set forth in SEQ ID NO: 19.

Antigen binding fragments of the anti-CRF antibodies described herein are also contemplated. The antigen binding fragment can be any part of an antibody that has at least one antigen binding site, and the antigen binding fragment may be part of a larger structure (an “antibody product”) that retains the ability of the antigen binding fragment to recognize CRF. For ease of reference, these antibody products that include antigen binding fragments are included in the disclosure herein of “antigen binding fragment.” Examples of antigen binding fragments, include, but are not limited to, Fab, F(ab′)₂, a monospecific or bispecific Fab₂, a trispecific Fab₃, scFv, dsFv, scFv-Fc, bispecific diabodies, trispecific triabodies, minibodies, a fragment of IgNAR (e.g., V-NAR), a fragment of hcIgG (e.g., VhH), bis-scFvs, fragments expressed by a Fab expression library, and the like. In exemplary aspects, the antigen binding fragment is a domain antibody, VhH domain, V-NAR domain, VH domain, VL domain, or the like. Antibody fragments of the disclosure, however, are not limited to these exemplary types of antibody fragments. In exemplary aspects, antigen binding fragment is a Fab fragment. In exemplary aspects, the antigen binding fragment comprises two Fab fragments. In exemplary aspects, the antigen binding fragment comprises two Fab fragments connected via a linker. In exemplary aspects, the antigen binding fragment comprises or is a minibody comprising two Fab fragments. In exemplary aspects, the antigen binding fragment comprises, or is, a minibody comprising two Fab fragments joined via a linker. Minibodies are known in the art. See, e.g., Hu et al., Cancer Res 56:3055-3061 (1996). In exemplary aspects, the antigen binding fragment comprises or is a minibody comprising two Fab fragments joined via a linker, optionally, comprising an alkaline phosphatase domain.

A domain antibody comprises a functional binding unit of an antibody, and can correspond to the variable regions of either the heavy (VH) or light (VL) chains of antibodies. A domain antibody can have a molecular weight of approximately 13 kDa, or approximately one-tenth of a full antibody. Domain antibodies may be derived from full antibodies such as those described herein.

In some embodiments, the scFv is attached to a human Fc domain. In some embodiments, the Fc domain does not activate Fc effector functions.

### Methods of Antibody or Antigen Binding Fragment Production

Suitable methods of making antibodies are known in the art. For instance, standard hybridoma methods are described in, e.g., Harlow and Lane (eds.), Antibodies: A Laboratory Manual, CSH Press (1988), and CA. Janeway et al. (eds.), Immunobiology, 5th Ed., Garland Publishing, New York, NY (2001)). Monoclonal antibodies for use in the methods of the disclosure may be prepared using any technique which provides for the production of antibody molecules by continuous cell lines in culture. These include but are not limited to the hybridoma technique originally described by Koehler and Milstein (Nature 256:495-497, 1975), the human B-cell hybridoma technique (Kosbor et al., Immunol Today 4:72, 1983; Cote et al., Proc Natl Acad Sci 80:2026-2030, 1983) and the EBV-hybridoma technique (Cole et al., Monoclonal Antibodies and Cancer Therapy, Alan R Liss Inc, New York N.Y., pp 77-96, (1985). Alternatively, other methods, such as EBV-hybridoma methods (Haskard and Archer, J. Immunol. Methods, 74 (2), 361-67 (1984), and Roder et al., Methods Enzymol., 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. Nos. 5,545,806, 5,569,825, and 5,714,352, and U.S. Patent Application Publication No. 2002/0197266 A1). Antibodies may also be produced by inducing in vivo production in the lymphocyte population or by screening recombinant immunoglobulin libraries or panels of highly specific binding reagents as disclosed in Orlandi et al (Proc Natl Acad Sci 86:3833-3837; 1989), and Winter G and Milstein C (Nature 349:293-299, 1991). If the full sequence of the antibody or antigen-binding fragment is known, then methods of producing recombinant proteins may be employed. See, e.g., “Protein production and purification” Nat Methods 5 (2): 135-146 (2008). In some embodiments, the antibodies (or antigen binding fragments) are isolated from cell culture or a biological sample if generated in vivo.

Phage display also can be used to generate the antibody of the present disclosures. In this regard, phage libraries encoding antigen-binding variable (V) domains of antibodies can be generated using standard molecular biology and recombinant DNA techniques (see, e.g., Sambrook et al. (eds.), Molecular Cloning, A Laboratory Manual, 3rd Edition, Cold Spring Harbor Laboratory Press, New York (2001)). Phage encoding a variable region with the desired specificity are selected for specific binding to the desired antigen, and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell (see, e.g., Janeway et al., supra, Huse et al., supra, and U.S. Pat. No. 6,265,150). Related methods also are described in U.S. Pat. Nos. 5,403,484; 5,571,698; 5,837,500; 5,702,892. The techniques described in U.S. Pat. Nos. 5,780,279; 5,821,047; 5,824,520; 5,855,885; 5,858,657; 5,871,907; 5,969,108; 6,057,098; and 6,225,447.

Antibodies can be produced by transgenic mice that are transgenic for specific heavy and light chain immunoglobulin genes. Such methods are known in the art and described in, for example U.S. Pat. Nos. 5,545,806 and 5,569,825, and Janeway et al., supra.

Methods for generating humanized antibodies are well known in the art and are described in detail in, for example, Janeway et al., supra, U.S. Pat. Nos. 5,225,539, 5,585,089 and 5,693,761, European Patent No. 0239400 BI, and United Kingdom Patent No. 2188638. Humanized antibodies can also be generated using the antibody resurfacing technology described in U.S. Pat. No. 5,639,641 and Pedersen et al., J. Mol. Biol, 235, 959-973 (1994). A preferred chimeric or humanized antibody has a human constant region, while the variable region, or at least a CDR, of the antibody is derived from a non-human species. Methods for humanizing non-human antibodies are well known in the art. (See U.S. Pat. Nos. 5,585,089, and 5,693,762.)

Techniques developed for the production of “chimeric antibodies,” e.g., the splicing of mouse antibody genes to human antibody genes to obtain a molecule with appropriate antigen specificity and biological activity, can be used (Morrison et al., Proc Natl Acad Sci 81:6851-6855 (1984); Neuberger et al., Nature 312:604-608 (1984); Takeda et al., Nature 314:452-454 (1985)). Alternatively, techniques described for the production of single chain antibodies (U.S. Pat. No. 4,946,778) can be adapted to produce CRF-specific single chain antibodies.

Likewise, using techniques known in the art to isolate CDRs, compositions comprising CDRs are generated. Compositions comprising one, two, and/or three CDRs of a heavy chain variable region or a light chain variable region of a monoclonal antibody can be generated. The CDRs of exemplary antibodies are provided herein as SEQ ID NOs: 4-9 and 12-17. Techniques for cloning and expressing nucleotide and polypeptide sequences are well-established in the art (see, e.g., Sambrook et al., Molecular Cloning: A Laboratory Manual, 2nd Edition, Cold Spring Harbor, New York (1989)). The amplified CDR sequences are ligated into an appropriate expression vector. The vector comprising one, two, three, four, five and/or six cloned CDRs optionally contains additional polypeptide encoding regions linked to the CDR.

Chemically constructed bispecific antibodies may be prepared by chemically cross-linking heterologous Fab or F(ab′)₂ fragments by means of chemicals such as heterobifunctional reagent succinimidyl-3-(2-pyridyldithiol)-propionate (SPDP, Pierce Chemicals, Rockford, III.). The Fab and F(ab′)₂ fragments can be obtained from intact antibody by digesting it with papain or pepsin, respectively (Karpovsky et al., J. Exp. Med. 160:1686-701 (1984); Titus et al., J. Immunol., 138:4018-22 (1987)).

Methods of testing antibodies for the ability to bind to an epitope of CRF, regardless of how the antibodies are produced, are known in the art and include, e.g., radioimmunoassay (RIA), ELISA, Western blot, immunoprecipitation, surface plasmon resonance (e.g., BIAcore), and competitive inhibition assays (see, e.g., Janeway et al., infra, and U.S. Patent Application Publication No. 2002/0197266).

Antibody fragments that contain the antigen binding, or idiotype, of the antibody molecule may be generated by techniques known in the art. For example, a F(ab′)₂ fragment may be produced by pepsin digestion of the antibody molecule; Fab′ fragments may be generated by reducing the disulfide bridges of the F(ab′)₂ fragment; and two Fab′ fragments which may be generated by treating the antibody molecule with papain and a reducing agent. The disclosure is not limited to enzymatic methods of generating antigen binding fragments; the antigen binding fragment may be a recombinant antigen binding fragment produced by expressing a polynucleotide encoding the fragment in a suitable host cell.

A single-chain variable region fragments (scFv), which consists of a truncated Fab fragment comprising the variable (V) domain of an antibody heavy chain linked to a V domain of an antibody light chain via a synthetic peptide, can be generated using routine recombinant DNA technology techniques (see, e.g., Janeway et al., supra). Similarly, disulfide-stabilized variable region fragments (dsFv) can be prepared by recombinant DNA technology (see, e.g., Reiter et al., Protein Engineering, 7, 697-704 (1994)).

Recombinant antibody fragments, e.g., scFvs, can also be engineered to assemble into stable multimeric oligomers of high binding avidity and specificity to different target antigens. Such diabodies (dimers), triabodies (trimers) or tetrabodies (tetramers) are well known in the art, see e.g., Kortt et al., Biomol Eng. 2001 18:95-108, (2001) and Todorovska et al., J Immunol Methods. 248:47-66, (2001).

### Therapeutic Methods

The antibodies or antigen binding fragments thereof described herein are useful for treating or preventing disorders associated with CRF dysregulation or hypothalamic-pituitary-adrenal (HPA) axis activation (e.g., obesity or a stress-related disorder). The HPA axis includes positive and negative feedback interactions among three endocrine glands: the hypothalamus, the pituitary gland, and the adrenal glands that form the neuroendocrine system. Hormones released by the endocrine glands control reactions to stress, regulation of body processes like digestion, the immune system, mood and emotions, sexuality and energy storage and expenditure.

The HPA axis is dysregulated in several psychiatric and neuropsychiatric diseases, as well as in alcoholism and stroke. Examples of HPA axis biomarkers include ACTH and cortisol. Cortisol inhibits secretion of corticotropin-releasing factor (CRF), resulting in feedback inhibition of ACTH secretion. Pharmacological HPA axis and glucocorticoid suppression influences body weight, body composition, glucose metabolism, and potentially visceral organ lipid content in a positive way. Suppression of the HPA axis can be an effective therapeutic tool in combating, at least partially, the metabolic risk factors consistent with excess adiposity and/or chronic stress associated with obesity, neurodegenerative disease, and aging. Thus, the antibodies or antigen binding fragments thereof described herein are useful in treating obesity, including for their ability to alter body composition (e.g., increasing muscle mass).

“Obesity” refers to a condition in which the body weight of a mammal exceeds medically recommended limits by at least about 20%, based upon age and skeletal size. Obesity is characterized by fat cell hypertrophy and hyperplasia. Obesity may be characterized by the presence of one or more obesity-related phenotypes, including, for example, increased body mass (as measured, for example, by body mass index, or “BMI”), altered anthropometry, basal metabolic rates, or total energy expenditure, chronic disruption of the energy balance, increased Fat Mass as determined, for example, by DEXA (Dexa Fat Mass percent), altered maximum oxygen use (VO2), high fat oxidation, high relative resting rate, glucose resistance, hyperlipidemia, insulin resistance, and hyperglycemia. See also, for example, Hopkinson et al. (1997) Am J Clin Nutr 65 (2): 432-8 and Butte et al. (1999) Am J Clin Nutr 69 (2): 299-307. In humans the World Health Organization (WHO) defines obesity as a body mass index (BMI) of 30 or higher. The CDC defines obesity in adults as a BMI of 30 or higher, and in children as a BMI at or above the 95th percentile.

Obesity is considered a condition in which excess body fat may put a person at health risk (see Barlow and Dietz, Pediatrics 102: E29, 1998; National Institutes of Health, Obes. Res. 6 (suppl. 2): 51S-209S, 1998). Excess body fat can result from an imbalance of energy intake and energy expenditure.

Obesity can be assessed in a variety of ways. In some embodiments, obesity is assessed by determining the Body Mass Index (BMI) of a subject. In some embodiments, an “overweight” individual exhibits a BMI of 25.0 kg/m² to 29.9 kg/m² (also referred to as grade I obesity). In various embodiments, an “obese” individual exhibits a BMI of 30 kg/m² or greater (also referred to as grade II obesity). Obesity may or may not be associated with insulin resistance.

In some embodiments, obesity is assessed by determining the waist circumference in the subject. In men a waist circumference of 102 cm or more is considered obese, while in women a waist circumference of 88 cm or more is considered obese.

By “treatment” or “treating” it is meant that at least an amelioration of one or more symptoms associated with retina-related disease afflicting the patient, where amelioration is used in a broad sense to refer to at least a reduction in the magnitude of a parameter, e.g., a symptom associated with the disease being treated. As such, treatment also includes situations where a pathological condition, or at least symptoms associated therewith, is completely inhibited, e.g., prevented from happening, or stopped, e.g., terminated, such that patient no longer suffers from the impairment, or at least the symptoms that characterize the impairment. In some instances, “treatment”, “treating” and the like refer to obtaining a desired pharmacologic and/or physiologic effect. The effect may be prophylactic in terms of completely or partially preventing a disease or symptom thereof, and/or delaying development of a disease or symptom thereof, and/or may be therapeutic in terms of a partial or complete cure for a disease and/or adverse effect attributable to the disease. “Treatment” may be any treatment of a disease in a subject, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. Treatment may result in a variety of different physical manifestations, e.g., modulation in gene expression, increased neurogenesis, rejuvenation of tissue or organs, etc. Treatment of ongoing disease, where the treatment stabilizes or reduces the undesirable clinical symptoms of the patient, occurs in some embodiments. Such treatment may be performed prior to complete loss of function in the affected tissues. The subject therapy may be administered during the symptomatic stage of the disease, and in some cases after the symptomatic stage of the disease. The term “preventing” does not mean that 100% inhibition of the disease.

As used herein, “delaying development” of obesity, or an obesity-related disease, means to defer, hinder, slow, retard, stabilize, and/or postpone development of the disease. This delay can be of varying lengths of time, depending on the history of the disorder and/or the medical profile of the individual being treated. As is evident to one skilled in the art, a sufficient or significant delay can, in effect, encompass prevention, in that the individual does not develop detectable disease. A method that “delays” development of disease is a method that reduces the extent of the disease in a given time frame, when compared to not using the method. Such comparisons are typically based on animal or clinical studies, using a statistically significant number of subjects, although this knowledge can be based upon anecdotal evidence. “Delaying development” can mean that the extent and/or undesirable clinical manifestations are lessened and/or time course of the progression is slowed or lengthened, as compared to not administering the agent. Thus the term also includes, but is not limited to, alleviation of symptoms, diminishment of extent of disease, stabilized (i.e., not worsening) state of disease, delay or slowing of disease progression, and remission (whether partial or total) whether detectable or undetectable.

### Combination Therapy

The concept of “combination therapy” is well exploited in current medical practice. Treatment of a pathology by combining two or more agents that target the same pathogen or biochemical pathway sometimes results in greater efficacy and diminished side effects relative to the use of the therapeutically relevant dose of each agent alone. In some cases, the efficacy of the drug combination is additive (the efficacy of the combination is approximately equal to the sum of the effects of each drug alone), but in other cases the effect can be synergistic (the efficacy of the combination is greater than the sum of the effects of each drug given alone). As used herein, the term “combination therapy” means the two compounds can be delivered in a simultaneous manner, e.g. concurrently, or wherein one of the compounds is administered first, followed by the second agent, e.g., sequentially. The desired result can be either a subjective relief of one or more symptoms or an objectively identifiable improvement in the recipient of the dosage. Concurrent administration of two therapeutic agents does not require that the agents be administered at the same time or by the same route, as long as there is an overlap in the time period during which the agents are exerting their therapeutic effect. Simultaneous or sequential administration is contemplated, as is administration on different days or weeks.

The antibodies (or antigen binding fragments thereof) may be combined with another therapeutic agent that is useful in the treatment of obesity or disorders associated with the development and progression of obesity, and obesity related disorders, such as, diabetes, atherosclerosis, hypertension, hyperlipidaemias, dyslipidaemias, and cardiovascular disease. In various embodiments, an anti-CRF antibody or antigen binding fragment thereof described herein is administered in combination with an additional therapeutic useful for treating a condition or disorder associated with HPA axis activation (e.g., obesity). In various aspects, the additional therapeutic agent includes, but is not limited to, a GLP1R agonist/co-agonist, an SGLT2 inhibitor, a losartan/neprilysin inhibitor drug, or bariatric/metabolic surgery. In some aspects, the additional therapeutic agent is a GLP-1/incretin mimetic, including but not limited to, semaglutide, tirzepatide, or cagrilintide.

### Pharmaceutical Compositions

Pharmaceutical compositions comprising an anti-CRF antibody or antigen-binding fragment thereof described herein are also contemplated. In some embodiments, the pharmaceutical composition contains formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine, proline, methionine or lysine); antimicrobials; antioxidants (such as reducing agents, oxygen/free-radical scavengers, and chelating agents (e.g., ascorbic acid, EDTA, sodium sulfite or sodium hydrogen-sulfite)); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter-ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

Selection of the particular formulation materials described herein may be driven by, for example, the intended route of administration, delivery format and desired dosage. See, for example, REMINGTON'S PHARMACEUTICAL SCIENCES, supra. The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and may further include sorbitol or a suitable substitute therefor. In certain embodiments, the composition may be prepared for storage by mixing the selected composition having the desired degree of purity with optional formulation agents (REMINGTON'S PHARMACEUTICAL SCIENCES, supra) in the form of a lyophilized cake or an aqueous solution. Further, in some embodiments, the antibody or (antigen binding fragment thereof) may be formulated as a lyophilizate using appropriate excipients such as sucrose.

The pharmaceutical compositions of the invention can be selected for parenteral delivery. Alternatively, the compositions may be selected for inhalation or for delivery through the digestive tract, such as orally. Preparation of such pharmaceutically acceptable compositions is within the skill of the art. The formulation components are present preferably in concentrations that are acceptable to the site of administration. In certain embodiments, buffers are used to maintain the composition at physiological pH or at a slightly lower pH, typically within a pH range of from about 5 to about 8.

When parenteral administration is contemplated, the composition may be provided in the form of a pyrogen-free, parenterally acceptable aqueous solution comprising the desired antibody or fragment in a pharmaceutically acceptable vehicle. A particularly suitable vehicle for parenteral injection is sterile distilled water in which the antibody or fragment is formulated as a sterile, isotonic solution, properly preserved. In certain embodiments, implantable drug delivery devices may be used to introduce the desired antibody (or antigen binding fragment thereof).

Additional pharmaceutical compositions will be evident to those skilled in the art, including formulations involving antigen binding proteins in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, International Patent Application No. PCT/US93/00829, which is incorporated by reference and describes controlled release of porous polymeric microparticles for delivery of pharmaceutical compositions. Sustained-release preparations may include semipermeable polymer matrices in the form of shaped articles, e.g., films, or microcapsules. Sustained release matrices may include polyesters, hydrogels, polylactides (as disclosed in U.S. Pat. No. 3,773,919 and European Patent Application Publication No. EP058481, each of which is incorporated by reference), copolymers of L-glutamic acid and gamma ethyl-L-glutamate (Sidman et al., 1983, Biopolymers 2:547-556), poly(2-hydroxyethyl-methacrylate) (Langer et al., 1981, J. Biomed. Mater. Res. 15:167-277 and Langer, 1982, Chem. Tech. 12:98-105), ethylene vinyl acetate (Langer et al., 1981, supra) or poly-D (−)-3-hydroxybutyric acid (European Patent Application Publication No. EP133988). Sustained release compositions may also include liposomes that can be prepared by any of several methods known in the art. See, e.g., Eppstein et al., 1985, Proc. Natl. Acad. Sci. U.S.A. 82:3688-3692; European Patent Application Publication Nos. EP036676; EP088046 and EP143949, incorporated by reference.

Embodiments of the antibody formulations can further comprise one or more preservatives.

The therapeutically effective amount of an antibody-containing pharmaceutical composition to be employed will depend, for example, upon the therapeutic context and objectives. One skilled in the art will appreciate that the appropriate dosage levels for treatment will vary depending, in part, upon the molecule delivered, the indication(s) for which the antibody is being used, the route of administration, and the size (body weight, body surface or organ size) and/or condition (the age and general health) of the patient.

Administration of the compositions described herein will be via any common route so long as the target tissue is available via that route. The pharmaceutical compositions may be introduced into the subject by any conventional method, e.g., by intravenous, intradermal, intramusclar, intramammary, intraperitoneal, intrathecal, intraocular, retrobulbar, intrapulmonary (e.g., term release); by oral, sublingual, nasal, anal, vaginal, or transdermal delivery, or by surgical implantation at a particular site, e.g., embedded under the splenic capsule, brain, or in the cornea. The treatment may consist of a single dose or a plurality of doses over a period of time.

The following Examples are provided to further illustrate aspects of the disclosure, and are not meant to constrain the disclosure to any particular application or theory of operation.

All publications, patents and patent applications cited in this specification are herein incorporated by reference as if each individual publication or patent application were specifically and individually indicated to be incorporated by reference.

### Examples

### Example 1—Generation of Anti-CRF Antibodies by Active Immunization

Wild type mice were injected subcutaneously with 200 μg of an N-terminal CRF peptide fragment (either Peptide A: SEEPPISLDLTFHLL (amino acids 1-15 of SEQ ID NO: 1; or SEQ ID NO: 2) or Peptide B: SEEPPISLDLTFHLLREVLEM (amino acids 1-21 of SEQ ID NO: 1; or SEQ ID NO: 3)) emulsified in 200 μL of Complete Freund's Adjuvant, followed by two boosts of 200 μg of peptide emulsified in Incomplete Freund's Adjuvant injected IP at two week intervals. Following the final boost, serum was collected and assayed for Anti-CRF titers via direct ELISA.

### Example 2—Anti-CRH Antibodies Suppressed HPA-Axis Output In Vivo

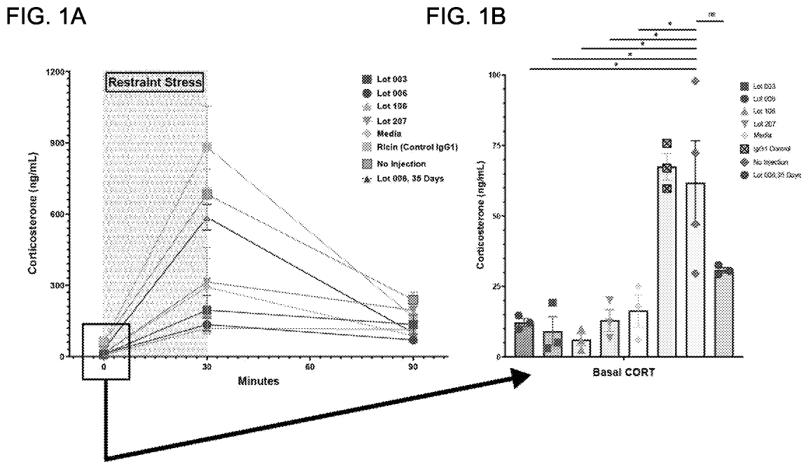

To ensure that the anti-CRF antibodies (e.g., Antibody B) effectively suppressed HPA-Axis output as measured by plasma corticosterone (CORT) levels, single-housed C57BL/6J×C3H/HeJ (hereby B6C3 mice) hybrid mice were injected with 20 mg/kg of Antibody B, an IgG1 control (anti-ricin IgG1), or a handling control. After 48 hours, animals were subjected to 30 minutes of restraint stress. All batches of Antibody B significantly reduced basal CORT and restraint-induced (FIG. 1A-1B). Even at 35 days post-injection, basal CORT trended towards suppression in animals receiving a single injection of Antibody B (FIG. 1B), although this did not reach statistical significance. Unpurified antibody media from a fiber cell system also significantly suppressed HPA-Axis output.

### Example 3—Anti-CRF Antibody Treatment Reduced Weight Gain and Improved Body Composition in Mice with Diet-Induced Obesity

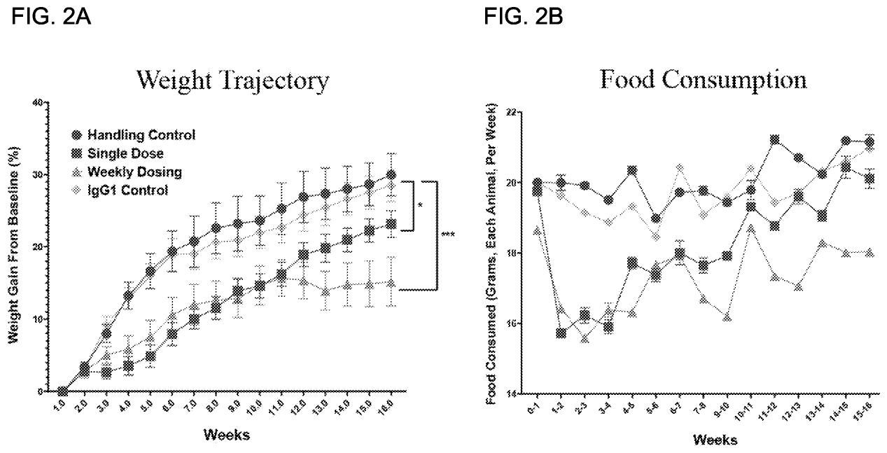

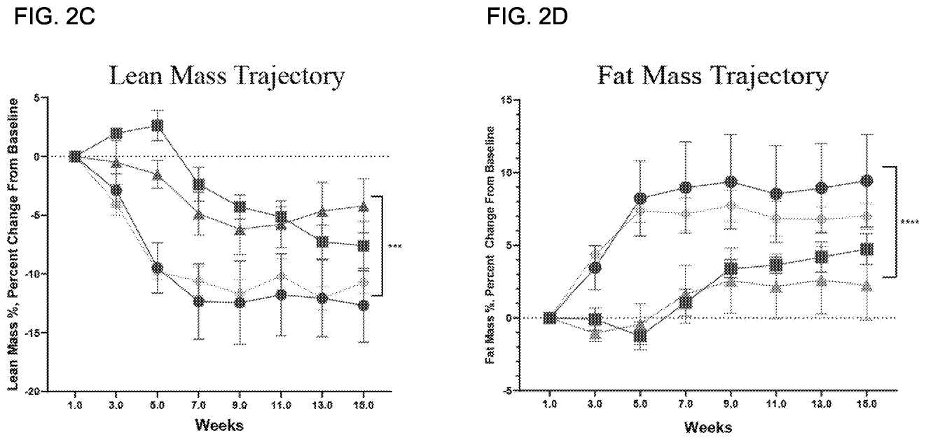

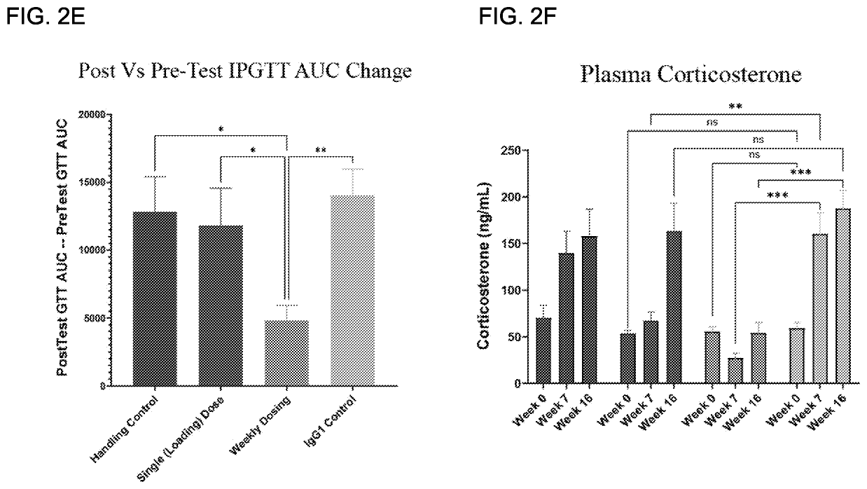

To determine if HPA-Axis suppression improved weight trajectory and body composition (primary endpoints), and to assess changes in glucose metabolism (secondary endpoint), 2-month-old C57BL6/J mice were placed on a high-fat diet (60% kcal) for 2 months. After 8 weeks, animals were injected with either weekly injections of Antibody B, a single dose of Antibody B followed by weekly injections of an IgG1 isotype-matched control antibody (anti-Ricin), weekly doses of an IgG1 control, or no treatment (handling control). Mouse IgG1 half-lives are approximately 6-7 days¹, so the meaningful pharmacological lifespan of injected doses is about 5-6 weeks, with waning efficacy over time. Indeed, animals that received Antibody B dosing demonstrated lower weight gain trajectories compared to other groups (FIG. 2, Table 1). Animals that received the single Antibody B dose had lower weight gain trajectories initially, but any the rate of weight gain mirrored that of the control cohorts after approximately 6 weeks after receiving a Antibody B dose. Pre-intervention and post-intervention intraperitoneal glucose tolerance tests (IPGTT) were conducted to assess cohort changes in glucose tolerance. Animals receiving weekly Antibody B injections had significantly less worsening of glucose handling than did other cohorts, including the single Antibody B injection group (Post-intervention minus pre-intervention IPGTT AUC: Weekly Dosing 4816.875+/−3163.059; Single Dose 11820+/−8660.577; Handling Control 12825+/−7742.743; IgG1 Control 14046.875+/−5525.222, n=10-11 all cohorts; Kruskal-Wallis One-Way ANOVA test with multiple comparisons, *=p<0.05, **=p<0.01). Animals that received weekly Antibody B doses held less fat mass and greater lean mess as a percentage of bodyweight at each point, and these differences were statistically significant at all timepoints after the 3-week timepoint. These time course differences in both fat mass and lean mass trajectories also reached statistically significance (RM ANOVA p<0.0001) (FIGS. 2C-2D). Plasma CORT levels were significantly lower in the animals receiving Antibody B weekly doses compared to control cohorts, while animals that received a single dose had significantly lower CORT value as 7 weeks (midpoint) compared to control cohorts. No groups differed in basal CORT levels prior to randomization (FIG. 2F). There was no statistical difference in adrenal or thymus weights at endpoint, although there was a slight trend towards lower adrenal weights and higher thymus weights (data not shown).

TABLE 1
Summary of Primary and Secondary Endpoints in B6 Male Mice

| | | | | | | | | Mean |
|---|---|---|---|---|---|---|---|---|
| | | | | | | | | Difference |
| | Handling | IgG1 | Single | Weekly | | | | (95% CI) (IgG1 |
| | Control | Control | Dose | Dose | | Statistical | | Control vs |
| Endpoint | (n = 9) | (n = 9) | (n = 9) | (n = 10) | Units | Test | p Value | Weekly Dose) |
| Weight | 29.987 +/− | 27.252 +/− | 23.751 +/− | 16.359 +/− | N/A | RM | <0.0001 | −10.892 |
| Change (% | 8.747 | 7.060 | 5.560 | 11.019 | | ANOVA | | (−14.388 to |
| of | | | | | | | | −7.397) |
| Baseline) | | | | | | | | P < 0.0001 |
| Percent Fat | 9.443 +/− | 7.000 +/− | 4.738 +/− | 2.050 +/− | N/A | RM | 0.0002 | −4.949 |
| Mass (% of | 3.181 | 0.888 | 1.056 | 2.108 | | ANOVA | | (−9.7174 to |
| Baseline) | | | | | | | | −0.1816) |
| | | | | | | | | P = 0.0428 |
| Percent | −9.134 +/− | −7.403 +/− | −4.651 +/− | −3.491 +/− | N/A | RM | <0.001 | 3.541 (0.1314 |
| Lean Mass | 2.633 | 0.651 | 1.323 | 1.890 | | ANOVA | | to 6.9504) |
| (% of | | | | | | | | P = 0.043 |
| Baseline) | | | | | | | | |
| Δ AUC, | 12825.0 +/− | 14046.9 +/− | 11820 +/− | 4816.9 +/− | mg/dL | One-Way | <0.05 | −9230.0 |
| IPGTT | 2580.91 | 1953.5 | 2738.72 | 1118.3 | | ANOVA | | (−13728 to |
| (Post-test − | | | | | | | | −4731.2) |
| pre-test) | | | | | | | | P = 0.0005 |

Plasma CORT

| | | | | | | | | |
|---|---|---|---|---|---|---|---|---|
| Pre- | 70.315 +/− | 59.217 +/− | 53.505 +/− | 55.828 +/− | ng/dL | RM | Ns | −3.389 |
| Intervention | 13.214 | 5.986 | 3.289 | 4.629 | | ANOVA | | (−18.154 to |
| | | | | | | | | 11.3764) |
| | | | | | | | | P = 0.633 |
| Midpoint | 129.993 +/− | 150.356 +/− | 67.180 +/− | 27.611 +/− | ng/dL | RM | <0.001 | −132.75 |
| | 13.214 | 22.358 | 9.312 | 4.607 | | ANOVA | | (−178.254 to |
| | | | | | | | | −87.235) |
| | | | | | | | | P < 0.0001 |
| Endpoint | 158.153 +/− | 187.757 +/− | 163.204 +/− | 54.443 +/− | ng/dL | RM | <0.0001 | −133.314 |
| | 69.769 | 53.128 | 95.148 | 28.026 | | ANOVA | | (−175.759 to |
| | | | | | | | | −90.869) |
| | | | | | | | | P < 0.0001 |

### Example 4—HPA-Axis Suppression Improved Several Metabolic Parameters in Both Male and Female Hybrid-Strain Mice

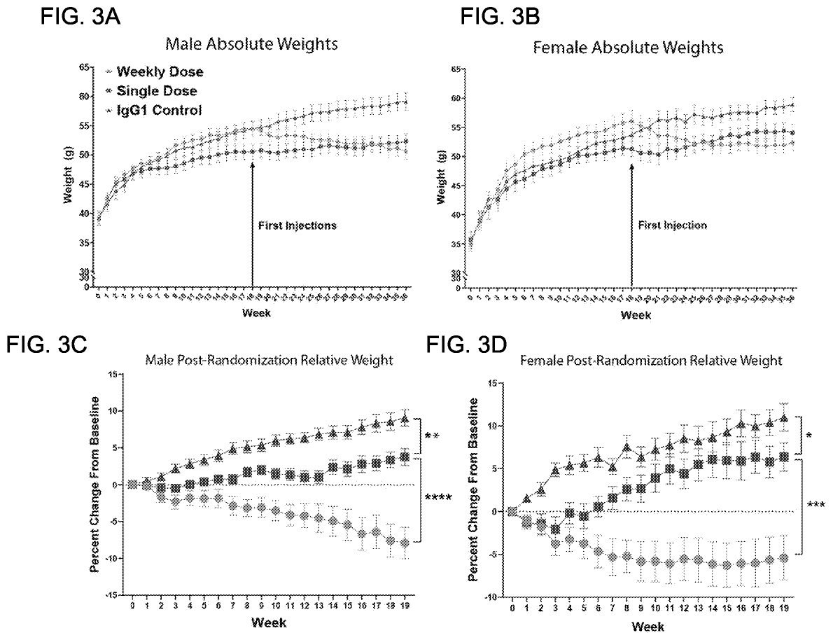

The Example 2 data indicated that HPA-Axis suppression had pleiotropic impacts on metabolism, including apparently via changes in food consumption behaviors, lean versus fat mass partitioning, and distribution of adipose tissue in an inbred strain of male mice (B6 mice). To determine if these findings were extrapolable, hybrid strain B6C3 male and female mice were placed on a high fat diet (HFD) (60% kcal fat) beginning at 7 months (29-30 weeks) of age. For 4 months, all animals were allowed to gain weight with no intervention. At 4 months, animals were randomized by cage to one of 3 treatment arms: 12.5 mg/kg of an IgG1 control antibody (anti-Ricin) weekly, a single 25 mg/kg dose of Antibody B followed by weekly 12.5 mg/kg doses of IgG1 control weekly, or a 25 mg/kg dose of Antibody B followed by weekly 12.5 mg/kg doses weekly. All animals received injections for 4 months (18 weeks). Animals were characterized prior to randomization, with no significant differences in weight (Males, Week of randomization: IgG1, 54.117 g+/−1.229; Single Dose, 50.505 g+/−1.332; Weekly Dose, 54.468 g+/−1.041; One-Way ANOVA with Brown-Forsythe/Welch correction and Dunnet correction for multiple comparison, P>0.10 for all comparisons. Note P=0.1162 for IgG1 control versus Single Dose), (Males, 1 week prior to randomization: IgG1, 53.774 g+/−1.253; Single Dose, 50.532 g+/−1.361; Weekly Dose, 54.152 g+/−1.087; One-Way ANOVA with Brown-Forsythe/Welch correction and Dunnet correction for multiple comparison, P>0.10 for all comparisons), (Females, Week of randomization: IgG1, 53.213 g+/−1.059; Single Dose, 51.371 g+/−1.218; Weekly Dose, 55.751 g+/−1.746; One-Way ANOVA with Brown-Forsythe/Welch correction and Dunnet correction for multiple comparison, P>0.05 for all comparisons.) (Females, 1 week prior to randomization: IgG1, 52.804 g+/−1.019; Single Dose, 51.086 g+/−1.229; Weekly Dose, 55.266 g+/−1.616; One-Way ANOVA with Brown-Forsythe/Welch correction and Dunnet correction for multiple comparison, P>0.10 for all comparisons) (FIGS. 3A-3B), body composition as assessed by EchoMRI, fasting (5 hrs) blood glucose, or plasma corticosterone (fasted 1 hr). Weight gain trajectories, both as a function of absolute weight and relative weight gained from point of randomization, were significantly higher in the IgG1 control cohorts (both male and female) compared to either Single Dose and Weekly dose cohorts, with the Weekly dosing cohort demonstrating significantly less weight gain (and actual weight loss) compared to the other 2 cohorts (Males, Repeat Measures ANOVA, p<0.0001; Females, Repeat Measure ANOVA, p<0.0001). At endpoint, weight gain relative to point of randomization was significantly higher in IgG1 controls versus all other cohorts, with significantly larger effects seen in Weekly dosing cohorts compared to Single dose cohorts (Males, 2 way ANOVA with Dunnett multiple comparisons test, IgG1 control vs Single dose, p<0.01; IgG1 control versus Weekly dose, p<0.0001, Weekly dose versus Single Dose, p<0.001); (Females, 2 way ANOVA with Dunnett multiple comparisons test, IgG1 control vs Single dose, p<0.05; IgG1 control versus Weekly dose, p<0.0001, Weekly dose versus Single Dose, p<0.01) (FIGS. 3C-3D, Tables 2 and 3).

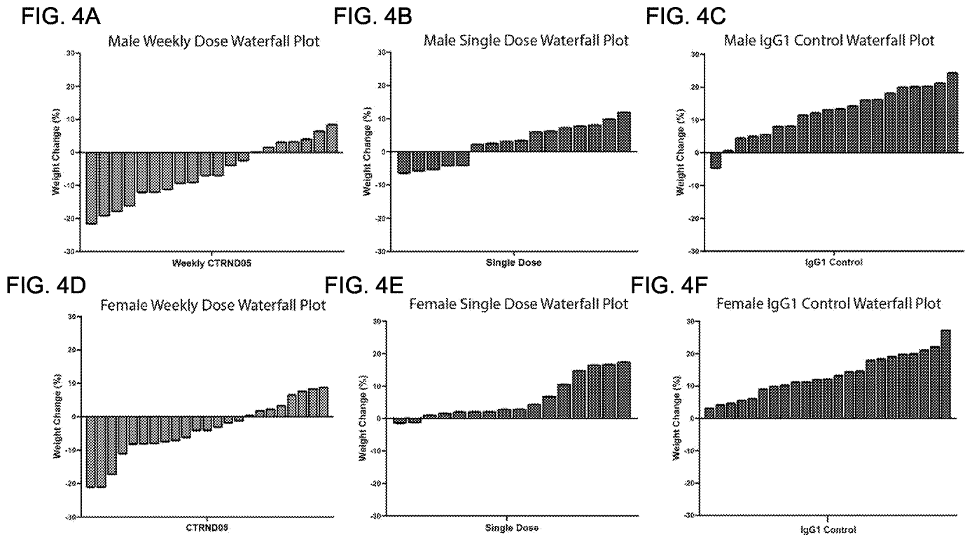

In both the male and female cohorts, animals in the Weekly dose cohort lost weight relative to their randomization starting point in 14/20 (males) and 15/21 (females) cases, ranging from losing 20% of their initial bodyweight to gaining about 10% of their initial weight. Single dose cohorts tended to gain weight by endpoint, especially in the case of the female cohort, ranging from a small amount of weight loss to approaching a 20% weight gain in the female cohort. In the IgG1 control cohort, all but one male and every female animal gained weight, ranging from very slight weight loss to gaining nearly 30% of randomization bodyweight. Distributions of weight change can be viewed as waterfall plots in FIG. 4. Body Composition was significantly improved in the Weekly dose cohorts on both an absolute mass and percentage of bodyweight basis (Males, Endpoint Percent Fat Mass, 2 Way ANOVA with Dunnett multiple comparison test, IgG1 vs Single Dose, p<0.05; IgG1 vs Weekly dose p<0.01; Single Dose vs Weekly dose p>0.10); (Males, Endpoint Percent Fat Mass, 2 way ANOVA with Dunnett multiple comparisons test, IgG1 vs Single Dose, p<0.01; IgG1 vs Weekly dose p<0.01; Single Dose vs Weekly dose p>0.10); Females, Endpoint Percent Fat Mass, 2 Way ANOVA with Dunnett multiple comparison test, IgG1 vs Single Dose, p<0.05; IgG1 vs Weekly dose p<0.01; Single Dose vs Weekly dose p>0.10); (Females, Endpoint Percent Fat Mass, 2 way ANOVA with Dunnett multiple comparisons test, IgG1 vs Single Dose, p<0.05; IgG1 vs Weekly dose p<0.001; Single Dose vs Weekly dose p>0.10).

TABLE 2
Primary and Secondary Endpoints in Male B6C3 Mice.

| | IgG1 | Single | Weekly | | | | Mean Difference (95% CI) |
|---|---|---|---|---|---|---|---|
| Endpoint | Control | Dose | Dose | | Statistical | | (IgG1 Control vs Weekly |
| Males | (n = 19) | (n = 14) | (n = 19) | Units | Test | P Value | Dose) |
| Weight | 9.080 +/− | 3.769 +/− | −7.913 +/− | N/A | RM | <0.0001 | −16.993 (−21.669 to −12.318) |
| Change (% of | 1.111 | 1.146 | 2.169 | | ANOVA | | P < 0.0001 |
| baseline) | | | | | | | |

Fat Mass

| | | | | | | | |
|---|---|---|---|---|---|---|---|
| Pre- | 31.383 +/− | 31.480 +/− | 31.532 +/− | N/A | One-Way | ns | 0.149 (−2.963 to 3.262) |
| Intervention | 1.186 | 1.846 | 1.713 | | ANOVA | | P > 0.10 |
| Percent Fat | | | | | | | |
| Mass (%) | | | | | | | |
| Post- | 33.361 +/− | 29.737 +/− | 25.345 +/− | N/A | One-Way | 0.0002 | −8.016 (−10.961 to −5.071) |
| Intervention | 0.884 | 1.849 | 1.713 | | ANOVA | | P < 0.001 |
| Percent Fat | | | | | | | |
| Mass (%) | | | | | | | |

Lean Mass

| | | | | | | | |
|---|---|---|---|---|---|---|---|
| Pre- | 62.137 +/− | 63.217 +/− | 63.137 +/− | N/A | One-Way | ns | 0.989 (−26.460 to 28.437) |
| Intervention | 1.068 | 1.533 | 1.940 | | ANOVA | | P > 0.10 |
| Percent Lean | | | | | | | |
| Mass (% of | | | | | | | |
| Baseline) | | | | | | | |
| Post- | 61.931 +/− | 65.680 +/− | 71.295 +/− | N/A | One-Way | <0.0001 | 9.364 (6.077 to 12.651) |
| Intervention | 1.086 | 1.720 | 1.940 | | ANOVA | | P < 0.0001 |
| Percent Lean | | | | | | | |
| Mass (% of | | | | | | | |
| Baseline) | | | | | | | |
| Subcutaneous | 1.946 +/− | 2.112 +/− | 2.994 +/− | N/A | One-Way | <0.0001 | 1.048 (0.6808 to 1.415) |
| Mesenteric | 0.100 | 0.099 | 0.139 | | ANOVA | | P < 0.0001 |
| Adipose Ratio | | | | | | | |
| Gross Liver | 4.027 +/− | 3.879 +/− | 3.143 +/− | g | One-Way | P = 0.0037 | −0.884 (−1.415 to −0.352) |
| Mass (G) | 0.173 | 0.256 | 0.177 | | ANOVA | | P = 0.0018 |
| Quadriceps | 8.77 +/− | 9.71 +/− | 10.24 +/− | mg/g | One-Way | ns | 1.470 (0.1014 to 2.8365) |
| Mass/BW | 0.557 | 0.404 | 0.399 | | ANOVA | (p = 0.069) | P = 0.036 |
| (mg/g) | | | | | | | |
| Gastrocnemius | 7.81 +/− | 8.89 +/− | 9.50 +/− | mg/g | One-Way | <0.01 | 1.690 (0.8221 to 2.5579) |
| Mass/BW | 0.223 | 0.333 | 0.378 | | ANOVA | | P = 0.003 |
| (mg/g) | | | | | | | |

Plasma CORT

| | | | | | | | |
|---|---|---|---|---|---|---|---|
| Pre- | 77.903 +/− | 78.476 +/− | 72.650 +/− | ng/dL | Two-Way | ns | −5.253 (−21.1256 to 10.6202) |
| Intervention | 7.435 | 11.157 | 10.694 | | ANOVA | | P = 0.503 |
| Midpoint | 118.582 +/− | 88.282 +/− | 30.230 +/− | ng/dL | Two-Way | P < 0.0001 | −88.351 (−119.1148 to |
| | 17.784 | 10.622 | 3.970 | | ANOVA | | −57.5880) |
| | | | | | | | P < 0.0001 |
| Endpoint | 118.798 +/− | 103.059 +/− | 52.358 +/− | ng/dL | Two-Way | P < 0.001 | −66.440 (−89.7185 to |
| | 10.416 | 16.050 | 7.810 | | ANOVA | | −43.1614) |
| | | | | | | | P < 0.0001 |

TABLE 3
Primary and Secondary Endpoints in Female B6C3 Mice.

| | IgG1 | Single | Weekly | | | | Mean Difference (95% CI) |
|---|---|---|---|---|---|---|---|
| Endpoint | Control | Dose | Dose | | Statistical | | (IgG1 Control vs Weekly |
| Female | (n = 19) | (n = 14) | (n = 19) | Units | Test | P Value | Dose) |
| Weight | 11.001 +/− | 6.732 +/− | −5.413 +/− | N/A | RM | <0.0001 | −16.414 (22.339 to |
| Change (% of | 1.613 | 1.638 | 2.600 | | ANOVA | | −10.489) |
| baseline) | | | | | | | P < 0.0001 |

Fat Mass

| | | | | | | | |
|---|---|---|---|---|---|---|---|
| Pre- | 41.655 +/− | 39.635 +/− | 43.291 +/− | N/A | One-Way | ns | 1.636 (−0.559 to 3.8313) |
| Intervention | 0.855 | 1.222 | 0.752 | | ANOVA | | P > 0.10 |
| Percent Fat | | | | | | | |
| Mass (%) | | | | | | | |
| Post- | 42.642 +/− | 37.946 +/− | 36.070 +/− | N/A | One-Way | P < 0.001 | −6.572 (−9.181 to −3.963) |
| Intervention | 0.858 | 1.117 | 1.328 | | ANOVA | | P < 0.0001 |
| Percent Fat | | | | | | | |
| Mass (%) | | | | | | | |

Lean Mass

| | | | | | | | |
|---|---|---|---|---|---|---|---|
| Pre- | 50.120 +/− | 54.074 +/− | 52.907 +/− | N/A | One-Way | ns | 2.787 (−0.559 to 6.133) |
| Intervention | 0.889 | 0.975 | 0.992 | | ANOVA | | P > 0.10 |
| Percent Lean | | | | | | | |
| Mass (% of | | | | | | | |
| Baseline) | | | | | | | |
| Post- | 61.931 +/− | 57.140 +/− | 59.782 +/− | N/A | One-Way | <0.0001 | 8.235 (5.260\ to 11.210) |
| Intervention | 1.086 | 1.601 | 1.569 | | ANOVA | | P < 0.0001 |
| Percent Lean | | | | | | | |
| Mass (% of | | | | | | | |
| Baseline) | | | | | | | |
| Subcutaneous | 1.856 +/− | 2.058 +/− | 2.999 +/− | N/A | One-Way | <0.0001 | 1.138 (0.5954 to 1.6802) |
| Mesenteric | 0.150 | 0.140 | 0.222 | | ANOVA | | P < 0.0001 |
| Adipose Ratio | | | | | | | |
| Gross Liver | 3.764 +/− | 3.786 +/− | 3.089 +/− | g | One-Way | ns | −0.675 (−1.316 to −0.336) |
| Mass (G) | 0.219 | 0.258 | 0.229 | | ANOVA | (p = 0.060) | P = 0.0397 |
| Quadriceps | 7.28 +/− | 8.68 +/− | 9.47 +/− | mg/g | One-Way | <0.001 | 2.190 (1.2675 to 3.1125) |
| Mass/BW | 0.301 | 0.405 | 0.352 | | ANOVA | | P < 0.001 |
| (mg/g) | | | | | | | |
| Gastrocnemius | 6.98 +/− | 7.55 +/− | 8.01 +/− | mg/g | One-Way | <0.05 | 1.030 (0.2616 to 1.964) |
| Mass/BW | 0.268 | 0.285 | 0.302 | | ANOVA | | P = 0.01 |
| (mg/g) | | | | | | | |

Plasma CORT

| | | | | | | | |
|---|---|---|---|---|---|---|---|
| Pre- | 100.170 +/− | 100.045 +/− | 96.312 +/− | ng/dL | Two-Way | ns | −3.858 (−28.4758 to |
| Intervention | 10.827 | 8.703 | 9.972 | | ANOVA | | 20.7601) |
| | | | | | | | P = 0.7506 |
| Midpoint | 133.508 +/− | 112.198 +/− | 39.135 +/− | ng/dL | Two-Way | P < 0.0001 | −94.373 (−118 . . . 9681 to |
| | 13.922 | 14.732 | 4.736 | | ANOVA | | −69.7788) |
| | | | | | | | P < 0.0001 |
| Endpoint | 134.798 +/− | 135.259 +/− | 52.797 +/− | ng/dL | Two-Way | P < 0.0001 | −82.001 (−102.2652 to |
| | 11.602 | 11.964 | 3.493 | | ANOVA | | −61.7365) |
| | | | | | | | P < 0.0001 |

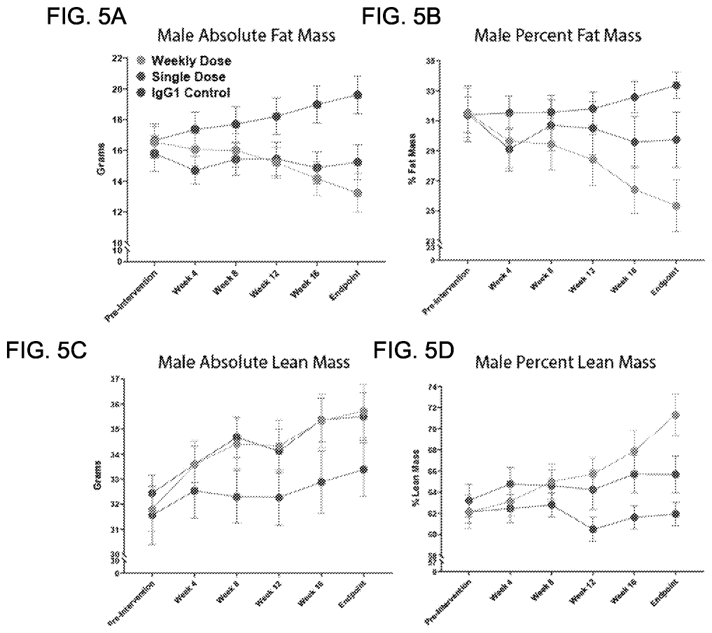

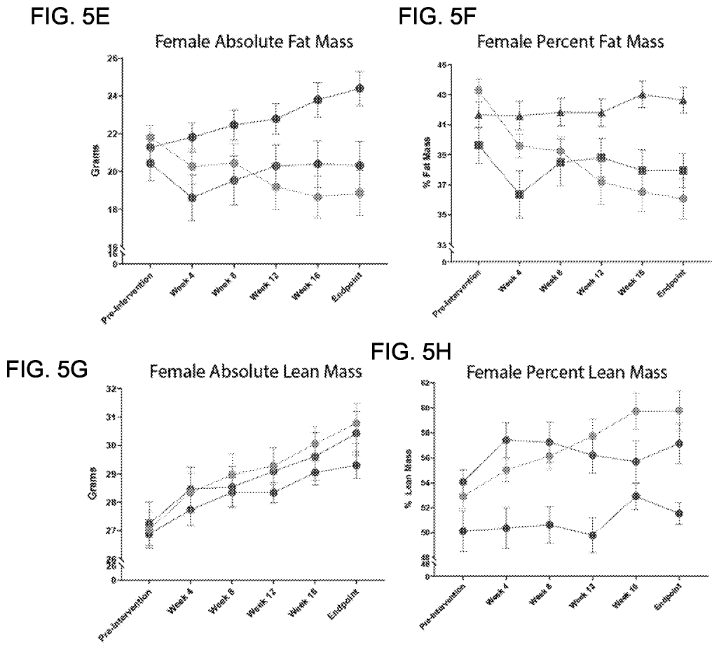

Repeat measure ANOVAs were significantly different for all cohorts for percent fat mass and lean mass changes over time. Both males and females that received weekly doses lost fat mass and gained lean mass on an absolute basis while decreasing total bodyweight on average, corresponding to higher relative amounts of lean mass. Male Single dose cohorts remained relatively stable in terms of weight, fat mass, and lean mass, seeing directional benefits at 1 month of treatment that had somewhat normalized by month 2. Female Single dose cohorts saw a similar trend in fat mass but weight gain and lean mass additions throughout the treatment phase. IgG1 control cohorts saw gains in weight, fat mass, and lean mass through the treatment phase, with relative levels of fat mass and lean mass remaining essentially constant (FIG. 5).

To assess the relative distribution of adipose tissue among cohorts, multiple fat pads at the experimental endpoint were weighed. Each fat pad tended to be smaller in the Weekly dose cohort relative to the Single dose or IgG1 control cohorts (data not shown). The ratio of the weight of the inguinal (subcutaneous) fat pad to the mesenteric (visceral) fat pad was significantly higher in the weekly dose cohorts (both sexes) (Brown-Forsythe ANOVA with multiple comparisons test, p<0.0001 both sexes), and this ratio did not significantly differ between the Single dose and IgG1 control cohorts (both sexes).

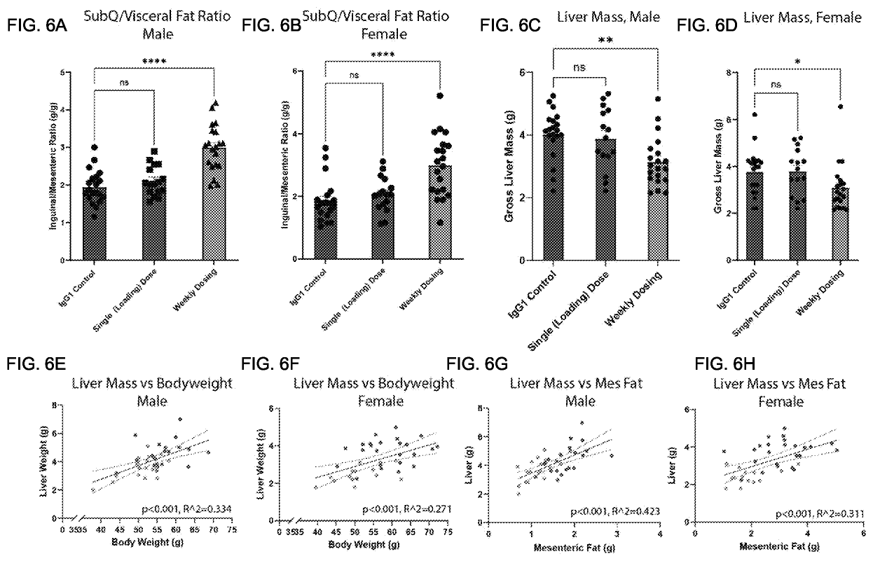

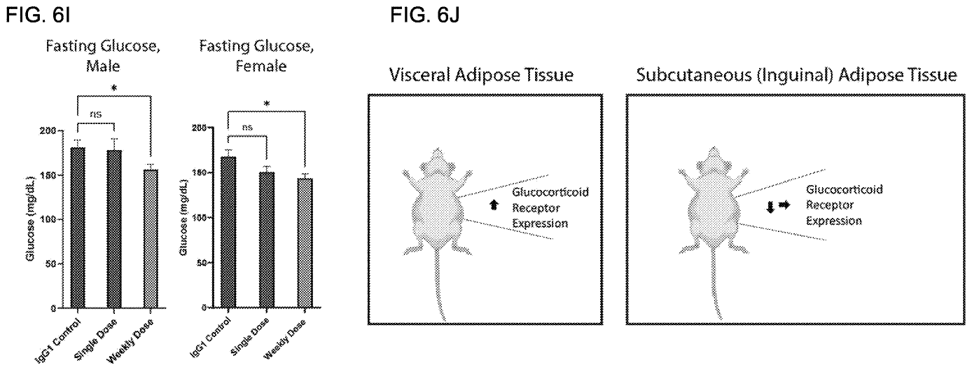

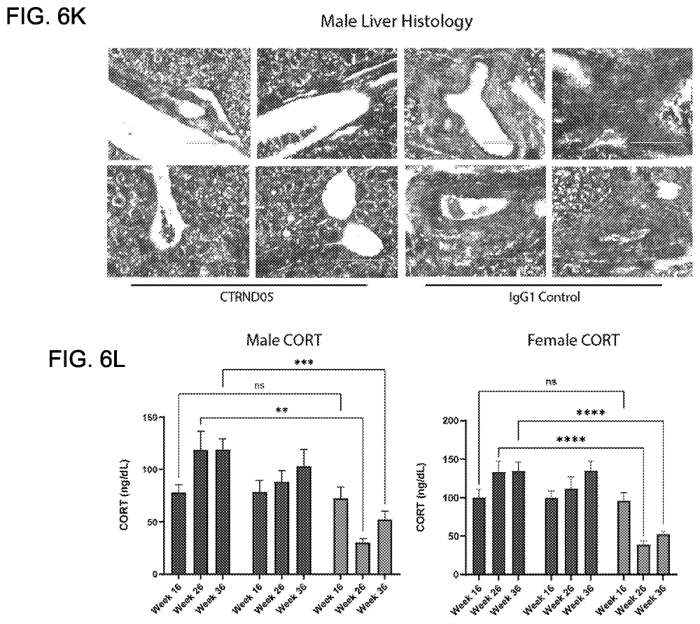

Consistent with a decrease in visceral adiposity, gross liver mass was lower in the Weekly dose cohorts (both sexes, Brown-Forsythe ANOVA with multiple comparisons test, Males Weekly dose vs Single Dose or IgG1 Control, p<0.01; Females Weekly dose versus Single Dose or IgG1 control, p<0.05). In both sexes, total mesenteric fat pad mass showed stronger correlations/was more predictive of variance in liver gross mass (FIGS. 6G-6H, R\hat{ }2 of 0.423 and 3.11 in males and females, respectively, p<0.001) than bodyweight (FIGS. 6E-6F, R\hat{ }2 of 0.334 and 0.271, respectively), mesenteric to subcutaneous fat pad mass ratio (not shown), or mesenteric fat pad mass relative to bodyweight (not shown), although the predictive difference between mesenteric fat mass and bodyweight was relatively small.

Fasting glucose was also modestly reduced in Weekly dose cohorts versus IgG1 controls (Both sexes, Brown-Forsythe ANOVA with multiple comparisons test, P<0.05), and fasting glucose was also significantly lower than the Single dose cohort in males (P<0.05) but not females (FIG. 6I), possibly due to increased liver mass and corresponding adiposity driving increased levels of basal gluconeogenesis.

To confirm that changes in parameters were proportional to plasma CORT levels, CORT levels were assessed prior to randomization, at treatment midpoint, and at the experimental endpoint. Indeed, in both sexes, while there was no difference in CORT levels prior to randomization (2 way ANOVA with Dunnett multiple comparison test, p>0.10), CORT was significantly lower in the Weekly dose cohorts versus either the Single Dose or IgG1 cohorts at both midpoint and endpoint (Males, Midpoint, IgG1 Control 118.582+/−17.784; Single dose 88.282+/−15.622; Weekly dose 30.230+/−3.970; 2 way ANOVA with Dunnett multiple comparison test, IgG1 control vs Weekly Dose, p<0.01; Single Dose vs IgG1 control, p=0.068); (Males, Endpoint, IgG1 Control 118+/−10.416; Single dose 103.059+/−16.050; Weekly dose 52.358+/−7.810; 2 way ANOVA with Dunnett multiple comparison test, IgG1 control vs Weekly Dose, p<0.001; Single Dose vs IgG1 control, p>0.10): (Females, Midpoint, IgG1 Control 133.582+/−13.922; Single dose 112.198+/−14.732; Weekly dose 39.135+/−4.736; 2 way ANOVA with Dunnett multiple comparison test, IgG1 control vs Weekly Dose, p<0.0001); (Females, Endpoint, IgG1 Control 134.798+/−11.602; Single dose 135.259+/−11.964; Weekly dose 52.797+/−3.493; 2 way ANOVA with Dunnett multiple comparison test, IgG1 control vs Weekly Dose, p<0.001 Single Dose vs Weekly Dose, p<0.05). Interestingly, CORT trended slightly lower in Single dose cohorts compared to IgG1 control at midpoint despite this being beyond the point where we would expect a pharmacologically relevant amount of antibody to be present. This effect disappeared by the end of the treatment phase.

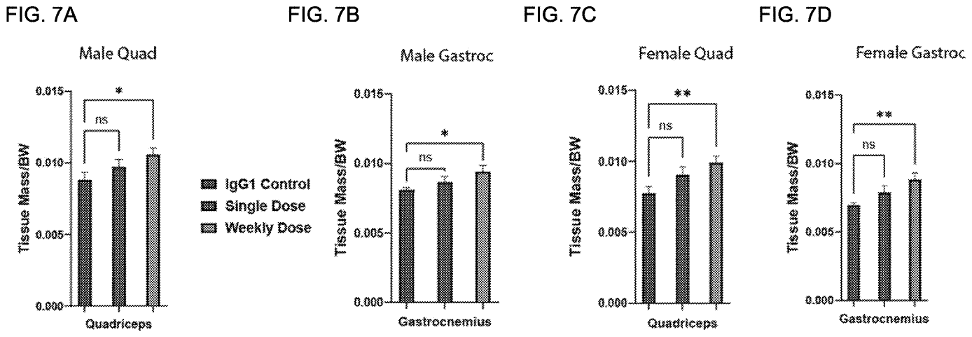

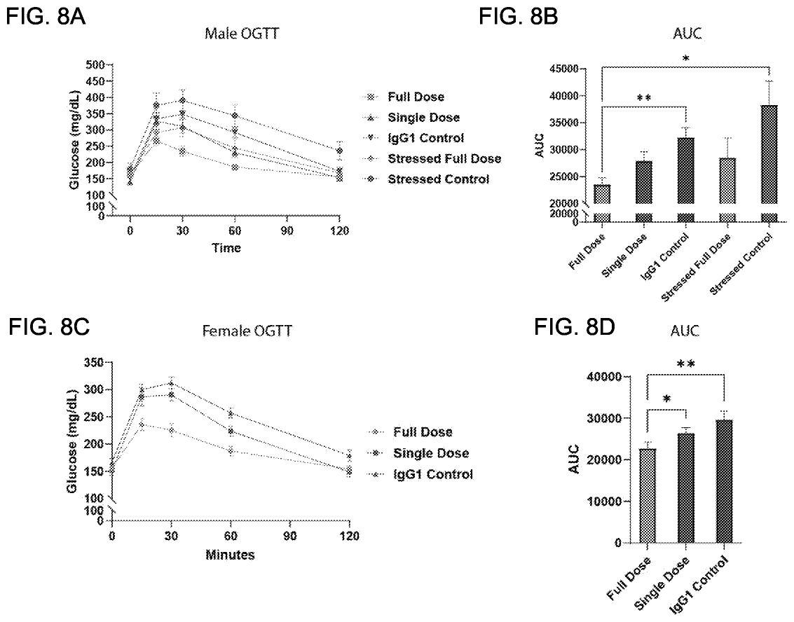

Finally, both quadriceps and gastrocnemius+soleus weights (normalized to bodyweight) were increased in Weekly dose cohorts compared to IgG1 control cohorts, with the Single dose cohort falling in between this cohorts without being statistically significantly different than either (Males, one-way ANOVA with Dunnett correction for multiple comparison test, IgG1 vs Weekly dose Quad, p<0.05, Gastroc, p<0.05; Female, Quad, p<0.01, Gastroc, p<0.01) (FIG. 7).

In order to assess glucose and insulin tolerance as a primary endpoint, 8-month-old male and female B6C3 mice were placed on HFD (60% kcal from fat) for 10 weeks. At that point, they were randomized to the same 3 cohorts: IgG1 Control, Single dose, or Weekly dose. In the males, an additional cohort consisting of a weekly dose group and a control group of animals was added, both of which were subjected to chronic stress (single housing+2-3 times weekly 30 minute restraint stress at a random time per day between Zeitberger time 10-24). Zeitberger (ZT) time is a normalized circadian timescale in which time zero is the first hour of the active phase, as in the first hour of darkness in murine animals and the first hour of light in humans. There were no significant differences in metabolic or weight parameters at the point of randomization between the groups prior to randomization.

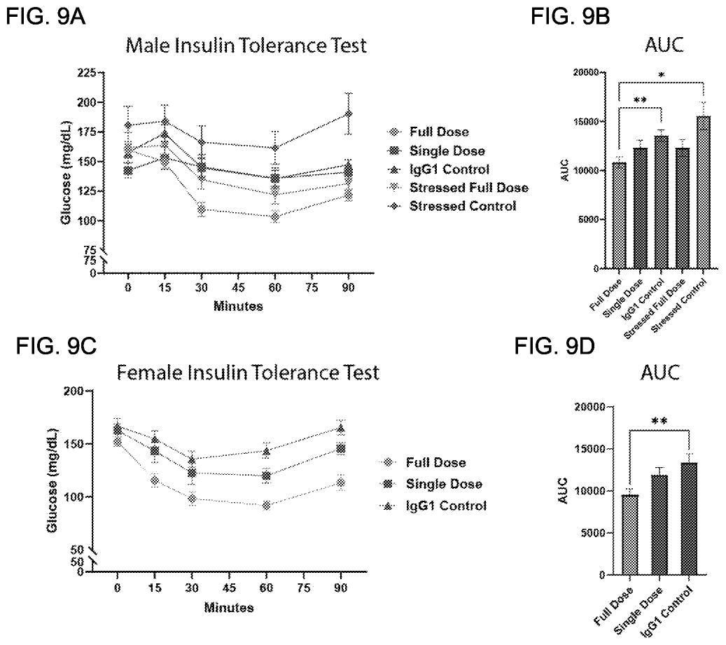

At endpoint, glucose tolerance tests and insulin tolerance tests were conducted in all cohorts (female cohort insulin tolerance tests not completed at time of writing). In both sexes, Weekly dose cohorts had significantly lower area under the curve (AUC) totals on an oral glucose tolerance test compared to IgG1 control. In females, the Weekly dose group also had significantly lower AUC compared to the Single dose cohort, but this was not the case in males. In males, the addition of the stress condition worsened glucose handling, but the effect was partially blunted by Antibody B treatment (FIG. 9).

In order to assess changes in gene expression changes in animals fed a high-fat diet for 8 months versus lean controls as well as how HPA-Axis suppression for 4 months versus IgG1 control, bulk RNA sequencing of the brain (no hypothalamus), hypothalamus, liver, gastrocnemius muscle, and mesenteric adipose tissue was conducted in both male and female mice. In the combined sex high-fat diet Antibody B vs IgG1 control cohorts, 87 differentially expressed genes (DEGs) were identified in the brain, 96 DEGs in the hypothalamus, 462 DEGs in the liver, 1524 DEGs in the gastrocnemius, and 26 DEGs in mesenteric fat. Threshold to be considered a DEG was a Padj<0.05. There was a significant amount of overlap between these results and transcriptomic analysis done previously in younger mice treated with Antibody B versus a control antibody 688. This included changes in genes involved in glucocorticoid signaling (SGK1), glucocorticoid receptor regulation (FKBP5), myelination in the brain (opalin), hypothalamic inflammation (SERPINA3n), muscle apelin signaling (apelin, apelin receptor), among others. Importantly, there was a decrease in POMC and PCSK1 expression in the hypothalamus of high-fat diet animals treated with Antibody B, supporting POMC and PCSK1 as compelling candidates driving several observations made herein. Several of these genes were altered in a sex-dependent fashion, where similar genes were differentially expressed, but the magnitude of difference varied between male and female animals.

In the comparison of animals that received high-fat diet versus a chow diet for 8 months (no anti-CRF antibody), 377 DEGs were identified in the brain, 1989 DEGs in the hypothalamus, 100 DEGs in the liver, 949 DEGs in the gastrocnemius, and 89 DEGs in mesenteric fat. Lipid metabolism genes seem to be overrepresented in several tissues, though gene ontology has not been completed at the time of this document's completion. Compellingly, several circadian genes are differentially expressed across several tissues, with BMAL1, PER2, and NR1D1 all showing some degree of difference in expression in skeletal muscle.

### Example 5—HPA-Axis Suppression Reduced Body Weight and Improved Body Composition in Leptin and MC4r Knockout Animals

In order to determine if glucocorticoid signaling in the arcuate nucleus of the hypothalamus (ARC) contributes to the observed impacts on weight, body composition, and metabolic outcomes, two well-characterized genetic models of obesity were employed: the MC4r and Leptin knockout mouse.

The leptin knockout mouse removes a central adipokine, leptin, that is an essential satiety signal that acts on leptin receptors on neurons that regulate consumptive behaviors and metabolic rate in the ARC 1323, meaning that primary signaling defect is upstream of the ARC from a cascade perspective. Leptin normally signals upstream of AgRP/NPY and POMC neuron projections to the PVN of the hypothalamus, among other satiety-regulating centers in the brain. This means that In contrast, the MCR4 knockout removes a receptor that canonical neurons (POMC and AgRP/NPY) in the ARC signal through—neurons that project to MC4r-containing neurons in the paraventricular nucleus of the hypothalamus (PVN) and which are stimulated (POMC, agonizes MC4r) or inhibited (AgRP/NPY, antagonizes MC4r). MC4r agonism decreases food intake and increases metabolic rate, potentially consistent with some of the effects we observed with HPA-Axis suppression. These two models conceptually represent monogenetic loss of satiety signals upstream (leptin) and downstream (MC4r) of ARC satiety and metabolism regulating neurons, which was hypothesized to enable testing of whether glucocorticoid suppression would prove less effective without the MC4r receptor to signal through. Therefore, if most of the impact of HPA-Axis suppression is due to sensitization to satiety and metabolic signals in the ARC by glucocorticoids, leptin knockout animals should still see some effect of HPA-Axis suppression, whereas MC4r knockout mice are expected to see minimal benefit due to the absence of a common input receptor.

To test this hypothesis, 6-week-old male and female knockout, heterozygous, and WT leptin and MC4r mice were randomized to receive either weekly injections of Antibody B or IgG1 control for 12 weeks (until 17 weeks of age).

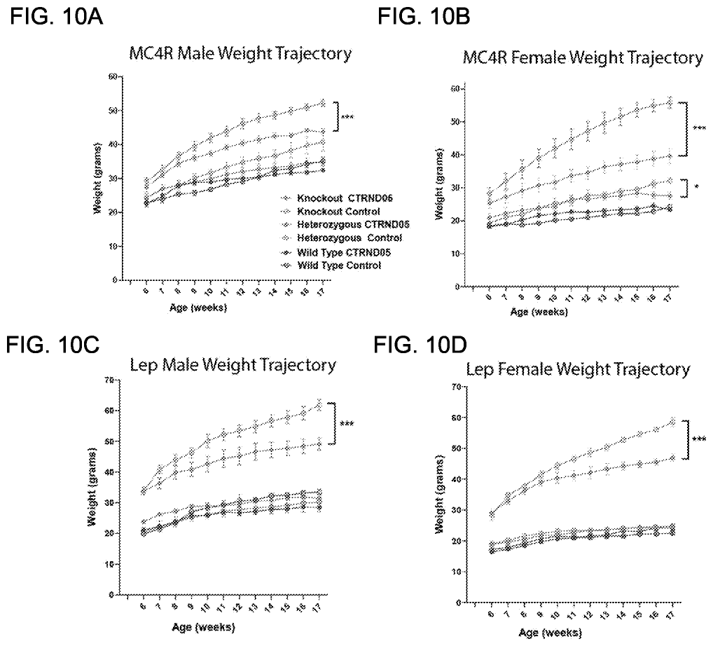

Indeed, significant weight reduction trajectories were observed in leptin knockout mice when treated with Antibody B (Males and females knockout cohorts, Repeated Measures ANOVA, Weekly dose vs IgG1 Control, p<0.001 to p<0.0001); (Males and Females knockout cohorts, 2/RM Way ANOVA with Dunnett multiple comparisons test, endpoint, p<0.0001 each cohort). Surprisingly, weight trajectories also improved in the MC4r knockout mice to essentially the same degree as the leptin knockout mice (FIG. 10). Further, significant improvements were observed in weight trajectory in MCR4 heterozygotes (Male and female MC4r heterozygotes, 2 Way ANOVA with Dunnett multiple comparison test, endpoint, p<0.05, both sexes) but not in the leptin heterozygotes (although this is expected based on the genetics data) 2. WT cohorts were not different with regard to weights after adjustment for multiple comparisons. Male Weekly dose groups were statistically heavier prior to adjustment for multiple comparisons compared to IgG1 controls prior to adjustment, which might be due to increases in lean mass seen in these cohorts (data not shown).

Fasted glucose levels generally paralleled the weight gain trajectories in the knockout cohorts, though leptin knockout cohorts did not reach statistical significance despite an approximately 20% difference in fasted glucose, likely due to being underpowered (Male and female MC4r knockouts, Brown-Forsythe ANOVA with multiple comparisons, Weekly dose vs IgG1 control, p<0.05; Leptin knockouts, p>0.05). Fasting glucose was also reduced in both male heterozygous Weekly dose cohorts compared to IgG1 cohorts (Brown-Forsythe ANOVA with multiple comparisons test, p<0.05). The female MC4r heterozygotes Weekly dose cohort trended towards an improvement in glucose tolerance relative to the IgG1 cohort but fell short of significance (p=0.0665) (data not shown).

Body composition was improved in all cohorts with Antibody B treatment. This includes increasing muscle mass in both absolute (not shown) and on a percentage of total bodyweight basis in the leptin KO mice, which are known to have reduced muscle mass that is corrected upon transplantation of WT adipocytes³. Unexpectedly, the body composition changes for both the MC4r and leptin heterozygotes was also quite large. All comparisons were statistically significant (Student's T-test with Welch correction and Bonferroni correction for 4 comparisons, p<0.01 to p<0.0001).

TABLE 4
Primary and Secondary Endpoints in Male MC4R Knockout Mice

| Endpoint | IgG1 | Weekly | | | | |
|---|---|---|---|---|---|---|
| MC4R KO | Control | Dose | | Statistical | | Mean Difference (95% CI) |
| Males | (n = 6) | (n = 6) | Units | Test | P Value | (IgG1 Control vs Weekly Dose) |
| Endpoint | 52.268 +/− | 43.713 +/− | N/A | RM | <0.0001 | −8.555 (−11.8070 to −5.3030) |
| Weight (g) | 1.048 | 1.016 | | ANOVA | | P = 0.0002 |
| Percent Fat | 37.892 +/− | 29.618 +/− | N/A | T-Test | <0.0001 | −8.274 (−10.7672 to −5.7806) |
| Mass, | 0.766 | 0.893 | | | | P < 0.0001 |
| Endpoint (%) | | | | | | |
| Percent Lean | 58.118 +/− | 67.191 +/− | N/A | T-Test | <0.001 | 9.073 (5.0103 to 13.1360) |
| Mass, | 1.158 | 1.396 | | | | P = 0.0007 |
| Endpoint (%) | | | | | | |
| Fasting | 1.946 +/− | 2.994 +/− | N/A | T-Test | <0.0001 | 1.048 (0.6808 to 1.415) |
| Glucose | 0.100 | 0.139 | | | | P < 0.0001 |
| (mg/dL) | | | | | | |
| Gross Liver | 4.027 +/− | 3.143 +/− | g | T-Test | P = 0.0037 | −1.629 (−2.3796 to −0.8786) |
| Mass (g) | 0.173 | 0.177 | | | | P = 0.0007 |
| Quadriceps | 3.729 +/− | 5.076 +/− | mg/g | T-Test | <0.001 | 1.347 (0.8746 to 1.8204) |
| Mass/BW | 0.195 | 0.176 | | | | P = 0.0001 |
| (mg/g) | | | | | | |
| Gastrocnemius | 2.998 +/− | 4.113 +/− | mg/g | T-Test | <0.01 | 1.115 (0.7712 to 1.4593) |
| Mass/BW | 0.118 | 0.142 | | | | P < 0.0001 |
| (mg/g) | | | | | | |

TABLE 5
Primary and Secondary Endpoints in Female MC4R Knockout Mice

| Endpoint | IgG1 | Weekly | | | | |
|---|---|---|---|---|---|---|
| MC4R KO | Control | Dose | | Statistical | | Mean Difference (95% CI) |
| Females | (n = 5) | (n = 6) | Units | Test | P Value | (IgG1 Control vs Weekly Dose) |
| Endpoint | 55.846 +/− | 39.657 +/− | N/A | RM | <0.001 | −16.189 (−22.1896 to −10.1891) |
| Weight (g) | 1.649 | 1.016 | | ANOVA | | P = 0.0001 |
| Percent Fat | 47.305 +/− | 32.985 +/− | N/A | T-Test | <0.0001 | −14.319 (−18.0454 to −10.5932) |
| Mass, | 0.712 | 1.372 | | | | P < 0.0001 |
| Endpoint (%) | | | | | | |
| Percent Lean | 46.388 +/− | 62.429 +/− | N/A | T-Test | <0.0001 | 16.042 (12.2628 to 19.8202) |
| Mass, | 0.834 | 1.349 | | | | P < 0.0001 |
| Endpoint (%) | | | | | | |
| Fasting | 203.400 +/− | 170.667 +/− | N/A | T-Test | <0.05 | −32.733 (−58.89.36 to −6.5731) |
| Glucose | 11.382 | 4.800 | | | | P = 0.0197 |
| (mg/dL) | | | | | | |
| Gross Liver | 5.263 +/− | 2.245 +/− | g | T-Test | P < 0.0001 | −3.018 (−3.9714 to −2.0638) |
| Mass (g) | 0.437 | 0.134 | | | | P = 0.0001 |
| Quadriceps | 1.937 +/− | 2.541 +/− | mg/g | T-Test | <0.05 | 0.604 (0.3045 to 0.9043) |
| Mass/BW | 0.071 | 0.105 | | | | P = 0.0014 |
| (mg/g) | | | | | | |
| Gastrocnemius | 1.598 +/− | 2.010 +/− | mg/g | T-Test | <0.05 | 0.412 (0.1988 to 0.6249) |
| Mass/BW | 0.045 | 0.080 | | | | P = 0.0018 |
| (mg/g) | | | | | | |

TABLE 6
Primary and Secondary Endpoints in Male Leptin Knockout Mice

| Endpoint | IgG1 | Weekly | | | | |
|---|---|---|---|---|---|---|
| Leptin KO | Control | Dose | | Statistical | | Mean Difference (95% CI) |
| Males | (n = 6) | (n = 7) | Units | Test | P Value | (IgG1 Control vs Weekly Dose) |
| Endpoint | 61.820 +/− | 49.139 +/− | N/A | RM | <0.001 | −12.681 (−19.1595 to −6.2033) |
| Weight (g) | 1.819 | 2.073 | | ANOVA | | P = 0.0014 |
| Percent Fat | 52.582 +/− | 42.330 +/− | N/A | T-Test | <0.0001 | −10.252 (−14.0403 to −6.4640) |
| Mass, | 0.998 | 1.165 | | | | P = 0.0001 |
| Endpoint (%) | | | | | | |
| Percent Lean | 41.125 +/− | 53.090 +/− | N/A | T-Test | <0.0001 | 11.964 (9.1194 to 14.8095) |
| Mass, | 0.760 | 1.062 | | | | P = 0.00577 |
| Endpoint (%) | | | | | | |
| Fasting | 208.600 +/− | 182.286 +/− | N/A | T-Test | ns | −26.314 (−53.6737 to 1.0451) |
| Glucose | 14.915 | 10.214 | | | | P > 0.05 |
| (mg/dL) | | | | | | |
| Gross Liver | 5.016 +/− | 3.964 +/− | g | T-Test | <0.05 | −1.052 (−2.1001 to −0.0039) |
| Mass (g) | 0.356 | 0.319 | | | | P = 0.0493 |
| Quadriceps | 3.025 +/− | 3.999 +/− | mg/g | T-Test | <0.01 | 0.974 (0.6165 to 1.3315) |
| Mass/BW | 0.125 | 0.171 | | | | P = 0.0001 |
| (mg/g) | | | | | | |
| Gastrocnemius | 2.410 +/− | 3.492 +/− | mg/g | T-Test | <0.01 | 1.082 (0.5708 to 1.5934) |
| Mass/BW | 0.148 | 0.549 | | | | P = 0.0007 |
| (mg/g) | | | | | | |

TABLE 7
Primary and Secondary Endpoints in Female Leptin Knockout Mice

| Endpoint | IgG1 | Weekly | | | | |
|---|---|---|---|---|---|---|
| Leptin KO | Control | Dose | | Statistical | | Mean Difference (95% CI) (IgG1 |
| Females | (n = 6) | (n = 7) | Units | Test | P Value | Control vs Weekly Dose) |
| Endpoint | 58.528 +/− | 46.906 +/− | N/A | RM | <0.0001 | −11.623 (−15.6770 to −7.5683) |
| Weight (g) | 1.504 | 1.121 | | ANOVA | | P = 0.0001 |
| Percent Fat | 59.785 +/− | 47.393 +/− | N/A | T-Test | <0.0001 | −12.391 (−13.6789 to −11.1040) |
| Mass, | 1.303 | 0.782 | | | | P < 0.0001 |
| Endpoint (%) | | | | | | |
| Percent Lean | 34.722 +/− | 49.575 +/− | N/A | T-Test | <0.001 | 14.853 (11.7239 to 17.9830) |
| Mass, | 1.179 | 0.471 | | | | P < 0.0001 |
| Endpoint (%) | | | | | | |
| Fasting | 206.500 +/− | 168.714 +/− | N/A | T-Test | <0.05 | −37.786 (−71.4764 to −4.0950) |
| Glucose | 13.892 | 7.773 | | | | P < 0.0001 |
| (mg/dL) | | | | | | |
| Gross Liver | 4.297 +/− | 3.444 +/− | g | T-Test | <0.05 | −0.853 (−1.6303 to −0.0756) |
| Mass (g) | 0.276 | 0.226 | | | | P = 0.0343 |
| Quadriceps | 1.517 +/− | 1.983 +/− | mg/g | T-Test | ns | 0.467 (0.2710 to 0.6818) |
| Mass/BW | 0.098 | 0.036 | | | | P = 0.003 |
| (mg/g) | | | | | | |
| Gastrocnemius | 1.350 +/− | 1.768 +/− | mg/g | T-Test | <0.05 | 0.418 (0.2548 to 0.5804) |
| Mass/BW | 0.073 | 0.045 | | | | P = 0.002 |
| (mg/g) | | | | | | |

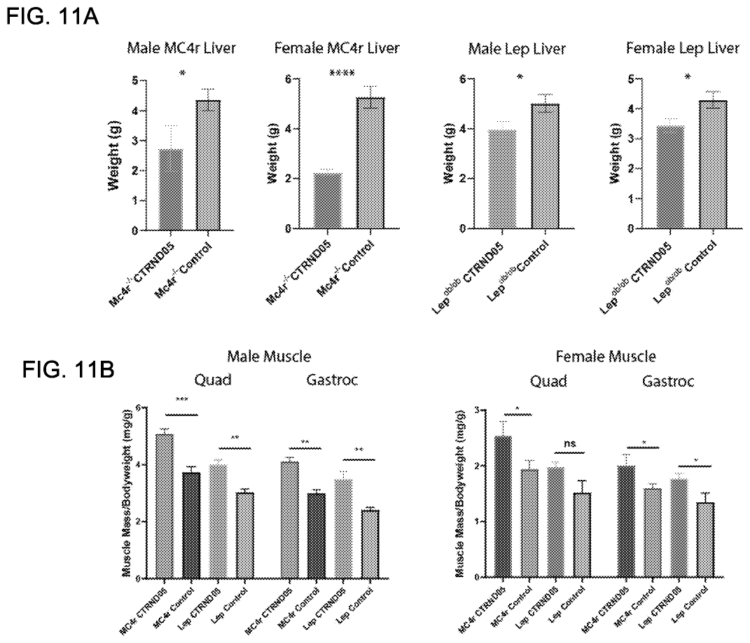

Consistent with the findings in wild-type mice, gross liver masses were reduced in all Weekly dose knockout cohorts compared to their corresponding IgG1 control cohort (Student T-tests, p<0.05 except for female MC4r, p<0.0001) (FIG. 11A). Gross weights of both gastrocnemius+soleus and quadriceps were statistically larger in Weekly dose cohorts compared to IgG1 except for the female leptin knockout quadriceps, which was not significantly different after correction for multiple comparisons (Student's T-test with Bonferroni correction, p<0.05 to p<0.001 except female leptin quadriceps) (FIG. 11B).

Again, similar to the data generated in wild-type mice, weekly dose cohorts had reduced fat pad mass in all fat pads collected, except for male gonadal fat pads, which were not significantly different (Brown-Forsythe ANOVA with multiple comparisons test, p<0.05 to p<0.0001, except for male leptin KO gonadal fat). There was also a statistically significant difference in the ratio value of the mesenteric to inguinal/subcutaneous fat pad mass in all knockouts except for the female leptin knockouts, though this was likely due to significant but roughly proportional reductions in the mass of both fat pads (Brown-Forsythe ANOVA with multiple comparisons test, p<0.05 to p<0.0001, except for female leptin KO gonadal fat) (data not shown).

### Example 6—HPA-Axis Suppression Reduced Body Weight and Improved Body Composition in Aged Male and Female Mice

Because metabolic dysfunction, broadly defined, tends to worsen with age⁴ in parallel with various indicators of resistance to glucocorticoid signaling⁵⁻⁷, Antibody B treatment was evaluated to determine whether it would provide similar weight trajectory and body composition effects in aged male and female B6C3 mice, which are genetically prone to weight gain with age (especially in females).

21-22-month-old mice were dosed with either 12.5 mg/kg Antibody B or IgG1 control for 6 weeks. Both sexes treated with Antibody B demonstrated significant weight loss over the course of 6 weeks (Both sexes, Repeat Measure ANOVA with Dunnett multiple comparison test, p<0.001).

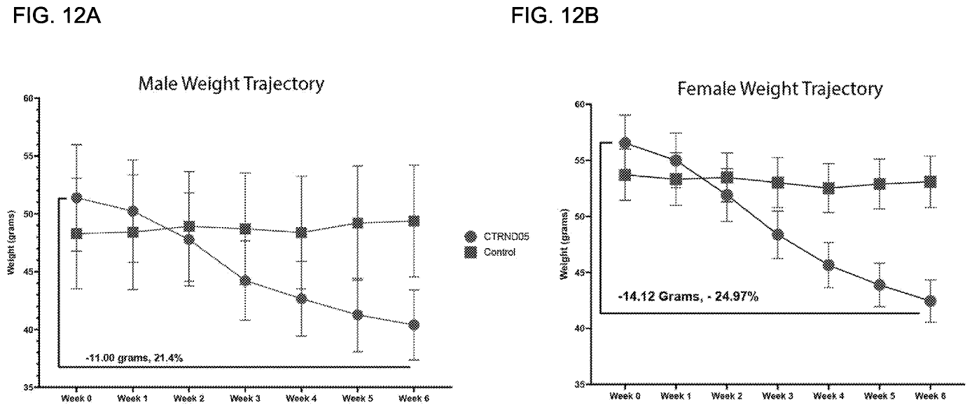

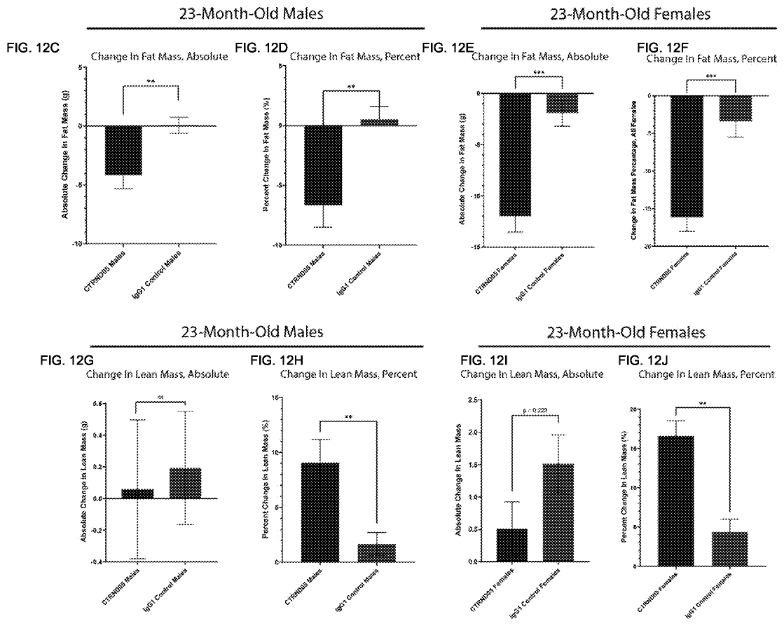

Body composition was also significantly improved in both sexes treated with Antibody B compared to IgG1 controls. In male mice, fat mass was reduced in absolute terms by nearly 4 grams in animals weighing approximately 51 grams on average at the time of randomization. In females, this corresponded to a roughly 11.5 gram loss in fat mass, corresponding to a loss of over 15% of fat mass during the treatment duration (Student T-tests with Bonferroni correction for multiple comparisons, p<0.01 in males, p<0.001 in females). Surprisingly, this occurred alongside an absolute gain in lean mass on average in both males and females treated with Antibody B, and this lean mass gain did not differ from the amount of gain seen in IgG1 treated animals (Student T-tests with Bonferroni correction for multiple comparisons). On a percent basis, this represents an approximately 8.5% and 16.5% increase in percent of body weight that is lean mass in Antibody B treated mice in males and females, respectively (FIG. 12).

### Example 7—HPA-Axis Suppression Appears to Modulate Stress-Induced Increases in Systolic Blood Pressure

Based in large part on long-standing, converging evidence identifying glucocorticoids as permissive agents that enable sympathetic nervous system-induced elevations in blood pressure (commonly co-occurring during stress), it was next evaluated whether suppression of glucocorticoid levels in a semi-acute paradigm could decrease stress-induced elevations in blood pressure brought upon by social defeat stress. Social defeat stress is a well-established chronic stress paradigm that is unique in that it combines both fear-based and social-status based stress components⁸⁻¹¹, and this paradigm has been demonstrated to impact cardiovascular indices¹². In this paradigm, unstressed animals saw no consistent alteration in their blood pressure or heart rate metrics from baseline following injections with either Antibody B or IgG1 control. Conversely, a modest decrease in systolic blood pressure is observed in animals treated with Antibody B, whereas animals that received a dose of the isotype-matched control realized no statistical difference and a slight overall increase in active phase blood pressure relative to their pre-injection window (data not shown).

### Example 8—Materials and Methods for Examples 2-7

Mouse Models. All experiments described herein were conducted according to the animal research guidelines from NIH. All mice used in experiments were housed in groups of 3-4 under a 12 hr reverse light/dark cycle. Animals were maintained at 22-23° C. No cages fell below 2 animals at any time during the studies with the exception of animals that were single housed intentionally to generate isolation stress. Animals were provided ad libitum access to either standard chow (Teklad 2918x) or high-fat diet consisting of 60% kcal from fat (Research Diets D12492i) as specified. For the pilot experiment, 48 C57BL/6J males (Jackson Laboratory, Strain No. 000664) were ordered at 8 weeks of age and placed on a high-fat diet in house throughout the experiment. Subsequent metabolic studies were conducted in male and female C57BL6×C3H/HeJ F1 or F2 generation outbred mice bred inhouse (breeders ordered from Jackson Laboratory, Strain No. 000664 and 000659, respectively) and were maintained on chow diets before being allocated to received chow of high-fat diet for the duration of the experiment at 29-30 weeks of age. Studies in aged mice were also conducted in male and female C57BL6×C3H/HeJ F1 or F2 generation outbred mice. Heterozygous leptin null mice were ordered from Jackson Laboratory (B6.Cg-Lep^{ob}/J+/−, Strain No. 000632) were bred inhouse and were maintained on a chow diet and randomized to a treatment group at 6 weeks of age. Heterozygous MC4r null mice were ordered from Jackson Laboratory (B6.129S4-Mc4^{tm1Lowl}/J+/−, Strain No. 032518) were also bred inhouse were maintained on a chow diet and randomized to a treatment group at 6 weeks of age. All animals in the study were euthanized by CO₂ inhalation and transcardial perfusion.

Mouse Experiment 1. 44 C57BL/6J (n=11 per cohort) male mice were given ad libitum access to high-fat diet (60% kcal fat) beginning at 2 months of age. After 8 weeks on high-fat diet (4 months of age), they were cluster randomized by cage to receive no treatment (handling control), saline control injection, a single 25 mg/kg of Antibody B, or a loading 25 mg/kg dose of Antibody B followed by weekly 12.5 mg/kg injections, with 11 animals in each group at the beginning of the experiment. They were maintained on high-fat diet until experimental endpoint at 8 months of age. Body composition was assessed every 2 weeks by EchoMRI (Echo Medical Systems, TX, USA) beginning at randomization (4 months of age). Blood collections were also conducted between ZT 20 and 24 at 4, 5.75, and 8 months to assess blood glucose, insulin, and corticosterone levels. Intraperitoneal glucose tolerance test was performed 1 week prior to euthanization. Animals were fasted for 4 hours prior to euthanization. Brain (including hypothalamus) and spleen were randomly allocated to be either snap frozen at −80° C. in liquid nitrogen or fixed in 10% formaldehyde for 36 hours prior to replacement of formaldehyde with PBS and maintained at 4° C. until analyzed. Liver, gastrocnemius, quadriceps, kidney, mesenteric fat, gonadal fat, subcutaneous (inguinal) fat, and spinal cord were all both frozen and fixed for all animals. Pituitary gland, adrenal glands, brown fat, heart, soleus were frozen for all animals. Thymus was fixed for all animals.

Mouse Experiment 2. Male and female C57BL6/J×C3H/HeJ mice were given ad libitum access to high-fat diet (60% kcal fat) or were continued on ad libitum chow diets (low-fat diet cohort) at 7 months (29-30 weeks) of age. At 11 months of age, animals were cluster randomized by cage (3:2:3 ratio) to receive a 12.5 mg/kg IgG1 control antibody injection weekly (anti-ricin antibody, produced by fiber cell and purified in house), a 25 mg/kg loading dose and a single 12.5 mg/kg maintenance dose of Antibody B followed by weekly 12.5 mg/kg IgG1 control injection, or a 25 mg/kg loading dose followed by weekly injections of Antibody B. These treatments were maintained until the experimental end at 15 months for all animals. Body composition was assessed prior to randomization (11 months of age) and then every 4 weeks thereafter (5 measures total). Blood collections were also conducted between ZT 20 and 24 at 11, 13, and 15 months to assess blood glucose, insulin, plasma lipid, inflammatory marker, and corticosterone levels. Animals were fasted for 4 hours prior to euthanization. In a 2:1 ratio, brain and hypothalamus were either snap frozen at −80° C. in liquid nitrogen separately or fixed together in 10% formaldehyde for 36 hours prior to replacement of formaldehyde with PBS and maintained at 4° C. until analyzed. Spleens and pancreas were either frozen or fixed in a 1:2 ratio. Liver, gastrocnemius, quadriceps, kidney, mesenteric fat, gonadal fat, subcutaneous (inguinal) fat, and spinal cord were all both frozen and fixed for all animals. Pituitary gland, adrenal glands, brown fat, heart, soleus were frozen for all animals. Thymus was fixed for all animals.

Mouse Experiment 3. Male and female C57BL6/J×C3H/HeJ mice were given ad libitum access to high-fat diet (60% kcal fat) at 8 months (33-34 weeks) of age. At 10.5 months of age, animals were cluster randomized by cage (2:1:2 ratio) to receive a 12.5 mg/kg IgG1 control antibody injection weekly (anti-ricin antibody, produced by fiber cell and purified in house), a 25 mg/kg loading dose and a single 12.5 mg/kg maintenance dose of Antibody B followed by weekly 12.5 mg/kg IgG1 control injection, or a 25 mg/kg loading dose followed by weekly injections of Antibody B. Two additional cohorts received either the IgG1 control or weekly Antibody B paradigms that were also individually housed and subjected to 3 bouts of restraint stress for 30 minutes at random times each week. These treatments were maintained until the experimental end at 13 months for all animals. Body composition was assessed prior to randomization (10.5 months of age) and then every 4 weeks thereafter (3 measures total). Blood collections were also conducted between ZT 20 and 24 at 11, 13, and 15 months to assess blood glucose, insulin, plasma lipid, inflammatory marker, and corticosterone levels. Animals were fasted for 4 hours prior to euthanization. Animals were randomized to receive either a saline injection or 1.5 U/kg Humalin R-100 insulin injection 15 minutes prior to euthanization in a 1:1 ratio. In a 2:1 ratio, brain and hypothalamus were either snap frozen at −80° C. in liquid nitrogen separately or fixed together in 10% formaldehyde for 36 hours prior to replacement of formaldehyde with PBS and maintained at 4° C. until analyzed. Spleens and pancreas were either frozen or fixed in a 1:2 ratio. Liver, gastrocnemius, quadriceps, soleus, kidney, mesenteric fat, gonadal fat, and subcutaneous (inguinal) fat were all both frozen and fixed for all animals. Pituitary gland, adrenal glands, and brown fat were frozen for all animals. Thymus, heart, and diaphragm were fixed for all animals.

Mouse Experiment 4. Wild type, heterozygous, and homozygous leptin deficient (B6.Cg-Lep^{ob}/J, hereby Ob/Ob) and MC4 receptor knockout (B6; 129S4-MC4^{tm1Lowl}/J, hereby MC4r) male and female breeders were order from Jackson Laboratory. Progeny mice were maintained on chow diets and cluster randomized by cage to receive either 12.5 mg/kg IgG1 control or Antibody B injections beginning at 6 weeks of age. Injections were given for 11 weeks until 17 weeks of age (4 months). Blood collections were also conducted between ZT 20 and 24 at endpoint (17 weeks) to assess blood glucose, insulin, plasma lipid, inflammatory marker, and corticosterone levels. Animals were fasted for 4 hours prior to euthanization. In a 2:1 ratio, brain and hypothalamus were either snap frozen at −80° C. in liquid nitrogen separately or fixed together in 10% formaldehyde for 36 hours prior to replacement of formaldehyde with PBS and maintained at 4° C. until analyzed. Spleens and pancreas were either frozen or fixed in a 1:2 ratio. Liver, gastrocnemius, quadriceps, kidney, mesenteric fat, gonadal fat, subcutaneous (inguinal) fat, and spinal cord were all both frozen and fixed for all animals. Pituitary gland, adrenal glands, brown fat, heart, soleus were frozen for all animals. Thymus was fixed for all animals.

Mouse Experiment 5. Aged Male and female C57BL6/J×C3H/HeJ mice were maintained on a chow diet throughout their lifespan. At 21-22 months of age, animals were cluster randomized by cage to receive a 12.5 mg/kg IgG1 control antibody injection weekly (anti-ricin antibody, produced by fiber cell and purified in house) or a 25 mg/kg loading dose followed by weekly injections of Antibody B. Injections were given for 6 weeks. Two additional cohorts received either the IgG1 control or weekly Antibody B paradigms and were subjected to 3 bouts of restraint stress for 30 minutes at random times each week (these animals were not individually housed). Body composition was assessed prior to randomization (20-21 months of age) and then at endpoint. Blood collections were also conducted between ZT 20 and 24 at randomization and endpoint to assess blood glucose, insulin, plasma lipid, inflammatory marker, and corticosterone levels. Animals were fasted for 4 hours prior to euthanization. In a 2:1 ratio, brain and hypothalamus were either snap frozen at −80° C. in liquid nitrogen separately or fixed together in 10% formaldehyde for 36 hours prior to replacement of formaldehyde with PBS and maintained at 4° C. until analyzed. The pancreas was either frozen or fixed in a 1:2 ratio. Liver, gastrocnemius, quadriceps, kidney, mesenteric fat, gonadal fat, subcutaneous (inguinal) fat, and spinal cord were all both frozen and fixed for all animals. Pituitary gland, adrenal glands, brown fat, heart, soleus were frozen for all animals. Thymus was fixed for all animals. Spleens were allocated for flow cytometry to assess the impact of glucocorticoid suppression on T cell composition and signature in aged mice.

Mouse Experiment 6. 3-month-old C57BL/6J male mice (n=4/cohort) were subjected to a social defeat stress paradigm in which they were placed in a cage with a dominant, aggressor mouse (CD1 retired breeder) for 20 seconds on 2 separate occasions. A matched cohort also were housed individually with no exposure to social defeat. Animals were subsequently housed in a common area separated by a permanent barrier such that the animals could see and smell each other, but they could not interact. Control animals were housed across from a similar-age C57BL6/J animal. 9-11 days after the initial social defeat stress bout, each animal's systolic blood pressure and heart rate were assessed by automated tail cuff (CODA8, Kent Scientific) over 24 hours. Baseline blood pressure was also measured prior to any initial stressor. Animals were subsequently treated with either 20 mg/kg Antibody B or IgG1 control. 24 hours later, blood pressure and heart rate were again measured over a 24-hour period. No animals experienced physical wounds that were deemed to be medically concerning as part of the stress paradigm.

Plasma Collection. All plasma draws were performed within two minutes of touching the cage and 45 seconds of contact with a mouse. A small (~1 mm) snip of the tip of the mouse tail was removed following wiping with ice-cold ethanol, and then 2-3 drops of blood (~20-30 μL) are drawn into a Sarstedt Microvette CB300 K2EDTA capillary plasma collection tube with 1× proteose inhibitor (complete protease inhibitor, Roche) added. The tail is then lightly cauterized to close the tail bleed. For multi-timepoint experiments samples, the scab is lightly removed at each subsequent time point time point. Tubes were kept on ice and then centrifuged at 3500×g for 12 minutes at 4° C. Supernatant plasma was then collected and stored at −80 until use.

Antibody Batch Verification. 4-month-old male C57BL6/J×C3H/HeJ were separated and single-housed and allowed to acclimate to single housing for 2 weeks. Animals were then injected with various antibody purification batches (n=3 animals per batch) or an IgG1 control (n=3 animals). 48 hours later, animals were subjected to a 30-minute restraint stress whereby the animals were placed in clear acrylic tubing and placed in their home cage for the duration of the stressor. Blood was collected via tail vein snip at 0, 30, 60 and 120 minutes for corticosterone measurement.

Corticosterone Measurement. MP Bio corticosterone double antibody RIA Kit (07120102) was used according to instructions. Mouse Plasma was diluted 1:100 in assay buffer for non-stress basal conditions, 1:200 for recovery timepoints, and 1:400 for max stress timepoints. Radioactivity assay portion was measured using a Wizard² automatic gamma counter (Revvity, MA, USA).

Glucose Tolerance Test. To measure glucose tolerance (GTT), mice were fasted for 5 hrs, and blood was collected from the tail vein immediately before and 15, 30, 60, and 120 min after i.p. injection (mouse experiment 1) or oral gavage (mouse experiment 3) of glucose (2 g/kg) to determine blood glucose concentrations. Blood was also collected at each timepoint for measure of endocrine (i.e. insulin) values. Blood glucose levels were measured in duplicate using 2 automated blood glucose readers (Agamatrix Jazz meter, Agamatrix, Salem, NH). Blood insulin levels were measured via ELISA (Crystal Chem, Illinois, US).

Insulin Tolerance Test. To measure peripheral insulin sensitivity (ITT), mice were fasted for 5 hrs, and blood glucose levels were measured before and at 15, 30, 60, and 90 minutes after i.p. injection of Humalin R U-100 insulin (1 U/kg) (Eli Lilly).

Echo MRI. Body composition was quantified in conscious mice on days 7, 28, and 42 post-treatment using EchoMRI Quantitative Magnetic Resonance Body Composition Analyzer (Echo Medical Systems, Houston, Texas).

Histology. For histological evaluation, formaldehyde-fixed, paraffin-embedded 5 micron were produced and stained sections were stained using hematoxylin and eosin (H&E), Masson's trichrome, and picrosirius red (liver).

Liver Pathology. Liver sections were evaluated, and grade and stage were determined by a pathologist experienced in liver diseases, using the NASH-Clinical Research Network criteria. Liver triacylglycerol (TAG) content was determined by extracting lipids and quantifying TAGs using the Infinity Triglycerides Reagent (Thermo Fisher Scientific; TR22421). Liver injury was evaluated by measuring plasma alanine aminotransferase (ALT) and aspartate aminotransferase (AST) activities (BQ-Kits BQ004A; GenWay GWB-BQK284).

Tissue Pulverization. A mortar and pestle was prechilled with liquid nitrogen. Brain, liver, mesenteric adipose, subcutaneous adipose, and gastrocnemius were placed in a small volume of liquid nitrogen and pulverized. The pulverized tissue was added to freezer-safe, RNAse free tubes for transcriptomic and proteomic analysis and stored at −80° C. until use.

RNA Extraction and RNA-seq. RNA was cleaned up over a Qiagen RNeasy column with on-column DNase treatment. A subsequent DNase treatment using Turbo DNA-free kit (AM1907; Thermo Fisher) was performed. RNA was quantified using the Qubit 4 fluorometer and the RNA HS assay (Q32852; Thermo Fisher). RNA quality was determined using the Fragment Analyzer Automated CE System and the Standard Sensitivity RNA Analysis kit (Agilent). 1 μg of total RNA was polyA enriched and subjected to library preparation using the TruSeq RNA Sample Prep Kit v2 (RS-122-2001; Illumina). Libraries were quantified using a library quantification kit (KAPA Biosystems). Library size was determined with the High-Sensitivity NGS Fragment Analysis Kit (Agilent). Libraries were pooled with a strategy to minimize batch effects from library preparation and sequencing and to achieve 30-50 million reads per sample. The resulting FASTQ files were aligned against the mouse genome using STAR alignment packages in R version 4.5.1. Differential gene expression analysis was performed with DESeq2. Changes in gene expression levels between treated versus control groups within each tissue were compared with “find DEGs.” WGCNA was performed to determine modules of genes with similar expression patterns across samples.

### Example 9—A Focus on the Role of Glucocorticoids on Metabolic Parameters in Mouse Models of Tau-Related Dementia

Experiments were designed to determine if glucocorticoid blockade could reduce the tau burden in the JNPL3 model in both lean and diet-induced obese animals, as well as to determine if metabolic parameters could be improved in the animals by glucocorticoid suppression.

PS19 tau transgenic mice were injected at post-natal day 0 (P0) with AAV-CRF or an empty capsid. It was observed that levels of phosphorylated tau, as detected by CP13 (pSer 202) staining, were potentially increased the hippocampus and hindbrain of AAV-CRF treated animals compared to controls at 12 weeks of age. This is earlier than the PS19 line has traditionally developed pathology, indicating that viral overexpression of CRF and/or the corresponding rise in plasma glucocorticoid levels may be driving this observed phenomena (data not shown).

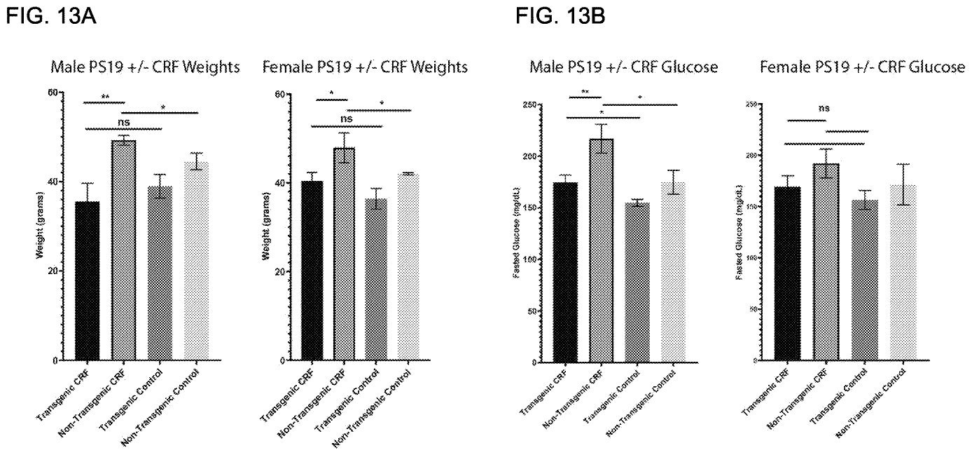

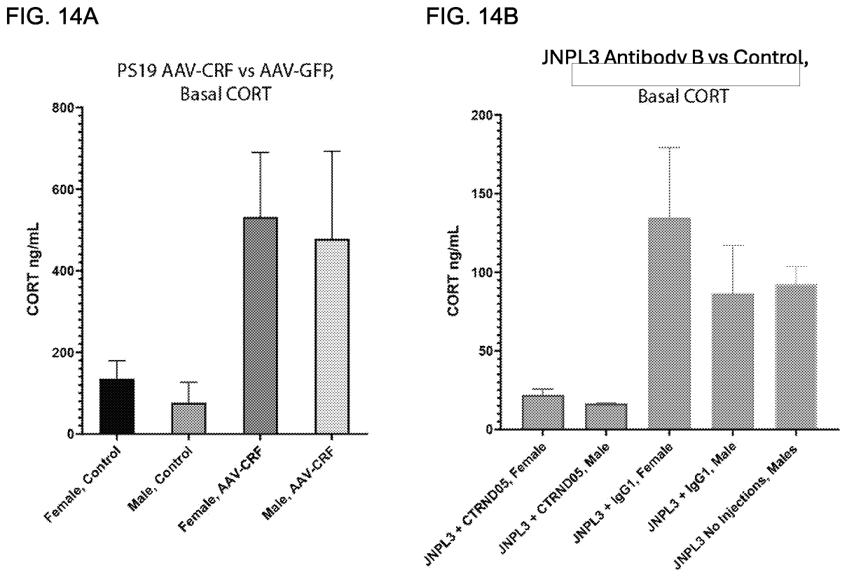

CRF over-expression had variable effects on weights, with PS19 male transgenic animals showing no or slightly reduced weight compared to capsid-injected transgenic animals (Brown-Forsythe ANOVA with multiple comparison test, p>0.10). In females, the opposite directional phenomenon was observed, though again the difference was not significant. Transgenic animals weighed significantly less than their non-transgenic counterparts in both sexes (Brown-Forsythe ANOVA with multiple comparison test, p<0.05 to p<0.01). In non-transgenic animals, CRF over-expression increases weight relative to controls (One-Way ANOVA with Brown-Forsythe/Welch correction and multiple comparison test, p<0.05, both sexes). Fasting glucose levels were significantly increased by CRF over-expression and decreased in transgenic animals in males. No statistical differences were observed in females, though these analyses were underpowered (FIG. 13). POAAV-CRF injection did achieve increased plasma CORT levels at 2.5 months in PS19 transgenic animals (FIG. 14A). In a different tau transgenic model—the JNPL3 P301L tau transgenic line—Antibody B treatment was successful in reducing plasma CORT levels in un-injected animals (FIG. 14B).

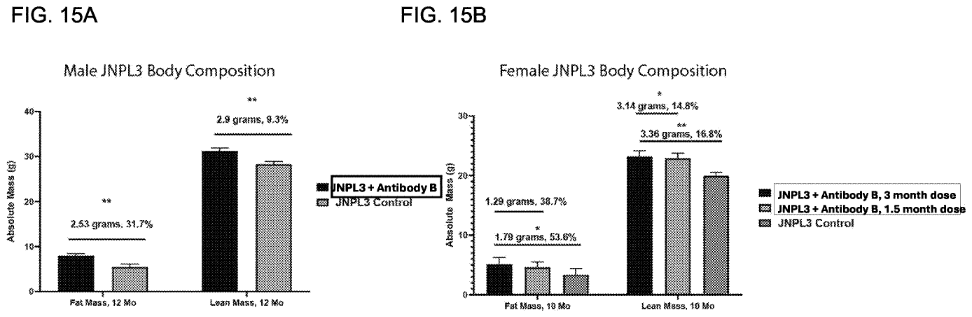

The homozygous JNPL3 line of tau transgenic animals is unique in that it tends to demonstrate significant weight loss and wasting in the latter stages of its lifespan, which is especially aggressive in females. To determine if we could see beneficial effects on metabolic parameters body composition and/or delay wasting in this line, JNPL3 chow-fed male and female mice were randomized to receive either Antibody B or IgG1 control for 3 months, beginning at 7 months in females and 9 months in males. This difference is due to the earlier-onset death curve seen in females. In females, an additional cohort was added that received 1.5 months of Antibody B followed by 1.5 months of IgG1 control antibody. In both males and females, 3 months of Antibody B treatment increased both lean and fat mass and preserved body mass (Student T-tests with Bonferroni correction for 2 comparisons, p<0.05 to p<0.01) (FIG. 15).

The data presented here argues that HPA-Axis and plasma glucocorticoid suppression are strongly associated with biologically relevant, pleotropic impacts on multiple systems in the body that culminate in beneficial metabolic changes in multiple tissues. This is consistent with historical data on the effects of adrenalectomy in combating various models of metabolic disease, but here there is no evidence of toxicity, organ failure, or necessity to supplement adrenal steroids.

### Materials and Methods

Mouse Models. All experiments presented in this study were conducted according to the animal research guidelines from NIH and were approved by the University of Florida IACUC. All mice used in experiments were housed in groups of 3-4 under a 12 hr reverse light/dark cycle. Animals were provided ad libitum access to either standard chow (Teklad 2918x) or high-fat diet consisting of 60% kcal from fat (Research Diets D12492i) as specified. Male and female Homozygous JNPL3 (Tg (Prnp-MAPT*P301L) JNPL3HImc) Tau transgenic mice were produced from homozygous breeders 1395. PS19 (B6; C3-Tg (Prnp-MAPT*P301S) PS19VIe/J) mice were bred inhouse from heterozygous breeders. Animals were fasted for 5 hours prior to any non-CORT blood draws. Animals were fasted for 4 hours prior to euthanization. All animals in the study were euthanized by CO₂ inhalation and transcardial perfusion.

Mouse Experiment 1. P0 Intracerebroventricular Injection. PS19 tau transgenic P0 pups were cryosanesthetized in tin foil on wet ice for 5 minutes. Two microliters containing of 1.0E13 of purified rAAV8-CRF was injected bilaterally into the lateral cerebral ventricles for a total of 4 μL per animal. Pups are then rewarmed and then placed back in the mother's cage.

Mouse Experiment 2. JNPL3 male and female mice were given ad libitum access to chow diet. At 7 months of age (females) and 9 months of age (males), animals were cluster randomized by cage to receive an IgG1 control injection (anti-ricin) or a loading 25 mg/kg dose of Antibody B followed by weekly 12.5 mg/kg injections. Experimental endpoints occurred after 3 months (females—10 months, males—12 months of age). A female cohort that received Antibody B injections from 6 months to 7.5 months was also included. Body composition was assessed prior to randomization (7/9 months of age), at midpoint (8.5/10.5 months), and at endpoint (10/12 months). Blood collections were also conducted between ZT 20 and 24 at 7/9 and 10/12 months to assess blood glucose, insulin, and corticosterone levels. Brains were hemisected and snap frozen and fixed. Spleens were randomly allocated to be either snap frozen at −80° C. in liquid nitrogen or fixed in 10% formaldehyde for 36 hours prior to replacement of formaldehyde with PBS and maintained at 4° C. until analyzed. Liver, gastrocnemius, quadriceps, kidney, mesenteric fat, gonadal fat, subcutaneous (inguinal) fat, and spinal cord were all both frozen and fixed for all animals. Pituitary gland, adrenal glands, and heart were frozen for all animals. Thymus was fixed for all animals.

Mouse Experiment 3. JNPL3 male and female mice were given ad libitum access to high-fat diet (60% kcal fat) beginning at 6/8 months of age through the duration of the experiment. At 6/8 months of age, they were cluster randomized by cage to receive either a 12.5 mg/kg IgG1 Control injection weekly or a loading 25 mg/kg dose of Antibody B followed by weekly 12.5 mg/kg injections. Body composition was assessed prior to randomization (6/8 months of age) and at endpoint (8/11 months). Blood collections were also conducted between ZT 20 and 24 at 6/8 and 9/11 months to assess blood glucose, insulin, and corticosterone levels. Animals were fasted for 4 hours prior to euthanization. Brains were hemisected and snap frozen and fixed. Spleens were randomly allocated to be either snap frozen at −80° C. in liquid nitrogen or fixed in 10% formaldehyde for 36 hours prior to replacement of formaldehyde with PBS and maintained at 4° C. until analyzed. Liver, gastrocnemius, quadriceps, kidney, mesenteric fat, gonadal fat, subcutaneous (inguinal) fat, and spinal cord were all both frozen and fixed for all animals. Pituitary gland, adrenal glands, and heart were frozen for all animals. Thymus was fixed for all animals.

### REFERENCES

1. Vieira, P. & Rajewsky, K. The half-lives of serum immunoglobulins in adult mice. Eur. J. Immunol. 18, 313-316 (1988).
2. Wade, K. H. et al. Loss-of-function mutations in the melanocortin 4 receptor in a UK birth cohort. Nat. Med. 27, 1088-1096 (2021).
3. Collins, K. H. et al. Leptin mediates the regulation of muscle mass and strength by adipose tissue. J. Physiol. 600, 3795-3817 (2022).
4. Hildrum, B., Mykletun, A., Hole, T., Midthjell, K. & Dahl, A. A. Age-specific prevalence of the metabolic syndrome defined by the International Diabetes Federation and the National Cholesterol Education Program: the Norwegian HUNT 2 study. BMC Public Health 7, 220 (2007).
5. Sabbagh, J. J. et al. Age-associated epigenetic upregulation of the FKBP5 gene selectively impairs stress resiliency. PloS One 9, e107241 (2014).
6. Kang, J. I., Kim, T. Y., Choi, J. H., So, H. S. & Kim, S. J. Allele-specific DNA methylation level of FKBP5 is associated with post-traumatic stress disorder. Psychoneuroendocrinology 103, 1-7 (2019).
7. Zannas, A. S. et al. Epigenetic upregulation of FKBP5 by aging and stress contributes to NF-κB-driven inflammation and cardiovascular risk. Proc. Natl. Acad. Sci. 116, 11370-11379 (2019).
8. Yoshida, K. et al. Chronic social defeat stress impairs goal-directed behavior through dysregulation of ventral hippocampal activity in male mice. Neuropsychopharmacol. Off. Publ. Am. Coll. Neuropsychopharmacol. 46, 1606-1616 (2021).
9. Venzala, E., García-García, A. L., Elizalde, N., Delagrange, P. & Tordera, R. M. Chronic social defeat stress model: behavioral features, antidepressant action, and interaction with biological risk factors. Psychopharmacology (Berl.) 224, 313-325 (2012).
10. Colyn, L., Venzala, E., Marco, S., Perez-Otaño, I. & Tordera, R. M. Chronic social defeat stress induces sustained synaptic structural changes in the prefrontal cortex and amygdala. Behav. Brain Res. 373, 112079 (2019).
11. Iñiguez, S. D. et al. Social defeat stress induces a depression-like phenotype in adolescent male c57BL/6 mice. Stress Amst. Neth. 17, 247-255 (2014).
12. Morais-Silva, G. et al. Cardiovascular outcomes related to social defeat stress: New insights from resilient and susceptible rats. Neurobiol. Stress 11, 100181 (2019). 1.

1. A method of decreasing cortisol in a subject in need thereof, comprising administering to the subject an antibody or antigen binding fragment thereof comprising a set of 6 CDRs set forth in SEQ ID NOs: 12-17 in an amount effective to reduce cortisol in the subject.

2. The method of claim 1, wherein the antibody comprises a heavy chain variable region comprising an amino acid sequence set forth in SEQ ID NO: 19.

3. The method of claim 1, wherein the antibody comprises a light chain variable region comprising an amino acid sequence set forth in SEQ ID NO: 18.

4. The method of claim 1, that is a humanized antibody.

5. The method of claim 1, that is an IgG.

6. The method of claim 1, wherein the antigen binding fragment is a Fab fragment or an scFv.

7. The method of claim 6, wherein the scFv comprises an amino acid sequence set forth in SEQ ID NO: 22 or SEQ ID NO: 23.

8. The method of claim 1, wherein the subject is obese.

9. A method of treating obesity in a subject in need thereof, comprising administering to the subject an antibody or antigen binding fragment thereof comprising a set of 6 CDRs set forth in SEQ ID NOs: 12-17.

10. The method of claim 9, wherein the antibody comprises a heavy chain variable region comprising an amino acid sequence set forth in SEQ ID NO: 19.

11. The method of claim 9, wherein the antibody comprises a light chain variable region comprising an amino acid sequence set forth in SEQ ID NO: 18.

12. The method of claim 9, that is a humanized antibody.

13. The method of claim 9, that is an IgG.

14. The method of claim 9, wherein the antigen binding fragment is a Fab fragment or an scFv.

15. The method of claim 14, wherein the scFv comprises an amino acid sequence set forth in SEQ ID NO: 22 or SEQ ID NO: 23.


SEQUENCE LISTING (23 sequences)

SEQ ID NO: 1 - protein, 41 aa, Homo sapiens
SEEPPISLDLTFHLLREVLEMARAEQLAQQAHSNRKLMEII

SEQ ID NO: 2 - protein, 15 aa, Homo sapiens
SEEPPISLDLTFHLL

SEQ ID NO: 3 - protein, 21 aa, Homo sapiens
SEEPPISLDLTFHLLREVLEM

SEQ ID NO: 4 - protein, 8 aa, Mus musculus
GFTFSSYA

SEQ ID NO: 5 - protein, 7 aa, Mus musculus
ISIGGST

SEQ ID NO: 6 - protein, 8 aa, Mus musculus
CARRGMDY

SEQ ID NO: 7 - protein, 16 aa, Mus musculus
RSSKSLLYKDGKTYLN

SEQ ID NO: 8 - protein, 6 aa, Mus musculus
LMSTRA

SEQ ID NO: 9 - protein, 13 aa, Mus musculus
QLVEYPLTFGAGT

SEQ ID NO: 10 - protein, 132 aa, Mus musculus
DIVITQDELSNPVTSGESVSISCRSSKSLLYKDGKTYLNWFLQRPGQSPQLLIYLMSTRASGVSDRFSGSGSGTDFTLEISRVKAEDVGVYYCQQLVEYPLTFGAGTKLELKRASGHHHHHHGSDYKDDDDK

SEQ ID NO: 11 - protein, 138 aa, Mus musculus
METDTLLLWVLLLWVPGSTGDGVQCEVKLVESGGGLVKPGGSLKLSCAASGFTFSSYAMSWVRQTPEKRLEWVASISIGGSTYYRDSVKGRCTISRDNAKNILYLQMRSLRSEDTAMYYCARRGMDYWGQGTSVTVSS

SEQ ID NO: 12 - protein, 8 aa, Mus musculus
GYSFTDST

SEQ ID NO: 13 - protein, 8 aa, Mus musculus
IHPDNGGT

SEQ ID NO: 14 - protein, 6 aa, Mus musculus
ANGFAY

SEQ ID NO: 15 - protein, 11 aa, Mus musculus
QSLLHSNGNTY

SEQ ID NO: 16 - skipped

SEQ ID NO: 17 - protein, 9 aa, Mus musculus
SQSTHVPLT

SEQ ID NO: 18 - protein, 112 aa, Mus musculus
DVVMTQTPLSLPVSLGDQASISCRSSQSLLHSNGNTYLHWYLQKPGQSPELLIYKVSNRFSGVPDRFSGSGSGTDFTLKISRVEAEDLGVYFCSQSTHVPLTFGAGTKLELK

SEQ ID NO: 19 - protein, 130 aa, Mus musculus
EVQLQQSGPELVKPGVSMKISCKASGYSFTDSTMNWVKQSHGKNLEWIGLIHPDNGGTIYNQKFKGKATLTVHKSSSTAYMELLSLTSEDSAVYYCANGFAYWGQGTLVTVSAAKTTPPSVYPLAPGSAA

SEQ ID NO: 20 - DNA, 336 bp, Mus musculus
  source 1..336: mol_type = other DNA
gatgtggtgatgacccagaccccgctgagcctgccggtgagcctgggcgatcaggcgagcattagctgccgcagcagccagagcctgctgcatagcaacggcaacacctatctgcattggtatctgcagaaaccgggccagagcccggaactgctgatttataaagtgagcaaccgctttagcggcgtgccggatcgctttagcggcagcggcagcggcaccgattttaccctgaaaattagccgcgtggaagcggaagatctgggcgtgtatttttgcagccagagcacccatgtgccgctgacctttggcgcgggcaccaaactggaactgaaa

SEQ ID NO: 21 - DNA, 342 bp, Mus musculus
  source 1..342: mol_type = other DNA
gaagtccagctccagcagagtggtcccgaactggttaagccgggagtgtctatgaagattagctgtaaggcatccggttactcttttacagactctacgatgaattgggtcaaacaatcccacgggaagaacttggagtggataggtctgatccaccctgataacggcggaacgatctacaaccagaagtttaaagggaaagcaaccctgactgtacacaagtcttcatctactgcgtacatggagttgctcagccttacctccgaggatagtgcagtttactactgtgctaacggtttcgcatactgggggcaaggaacgctcgtgacggtttcagccgcg

SEQ ID NO: 22 - protein, 285 aa, Mus musculus
METDTLLLWVLLLWVPGSTGDGVQCEVKLVESGGGLVKPGGSLKLSCAASGFTFSSYAMSWVRQTPEKRLEWVASISIGGSTYYRDSVKGRCTISRDNAKNILYLQMRSLRSEDTAMYYCARRGMDYWGQGTSVTVSSGGGGSGGGGSGGGGSDIVITQDELSNPVTSGESVSISCRSSKSLLYKDGKTYLNWFLQRPGQSPQLLIYLMSTRASGVSDRFSGSGSGTDFTLEISRVKAEDVGVYYCQQLVEYPLTFGAGTKLELKRASGHHHHHHGSDYKDDDDK

SEQ ID NO: 23 - protein, 296 aa, Mus musculus
METDTLLLWVLLLWVPGSTGDEVQLQQSGPELVKPGVSMKISCKASGYSFTDSTMNWVKQSHGKNLEWIGLIHPDNGGTIYNQKFKGKATLTVHKSSSTAYMELLSLTSEDSAVYYCANGFAYWGQGTLVTVSAAKTTPPSVYPLAPGSAAGGGGSGGGGSGGGGSDVVMTQTPLSLPVSLGDQASISCRSSQSLLHSNGNTYLHWYLQKPGQSPELLIYKVSNRFSGVPDRFSGSGSGTDFTLKISRVEAEDLGVYFCSQSTHVPLTFGAGTKLELKGSHHHHHHGSDYKDDDDK